(19) United States
(12) Patent Application Publication
(10) Pub. No.: US 2005/0019847 A1
(43) Pub. Date: Jan. 27, 2005

(54) ISG15-conjugated proteins

(76) Inventors: Dong-Er Zhang, San Diego, CA (US); Mikhail P. Malakhov, San Diego, CA (US); Oxana A. Malakhova, San Diego, CA (US)

Correspondence Address: BAKER & BOTTS, 30 ROCKEFELLER PLAZA, NEW YORK, NY, 10112, US

(21) Appl. No.: 10/771,680

(22) Filed: Feb. 3, 2004

Related U.S. Application Data
(60) Provisional application No. 60/444,939, filed on Feb. 3, 2003.

(51) Int. Cl. G01N 33/53

(52) U.S. Cl. 435/7.93

(57) ABSTRACT

The present invention provides methods to identify ISG15 target proteins, methods to identify compounds that alter the conjugation of ISG15 with target proteins, methods for altering the level of ISG15-conjugated protein in a patient or a sample, methods to diagnose a patient having a malcondition characterized by an altered level of ISG15-conjugated protein, ISG15-conjugates, methods to treat cancer by promoting conjugation of a target protein to ISG15, and antibodies that selectively bind to ISG15-conjugates.

### FIELD OF THE INVENTION

The present invention provides ISG15-conjugates, antibodies that selectively bind to ISG15-conjugates, methods to identify ISG15-target proteins, methods to identify compounds that alter the conjugation of ISG15 with target proteins, methods to alter the level of ISG15-conjugated protein in a patient or in a sample, methods to predict a patient's responsiveness to interferon treatment, and methods to diagnose a patient having a malcondition characterized by an altered level of ISG15-conjugated protein.

### BACKGROUND OF THE INVENTION

ISG15 is one of the most strongly induced genes after interferon (IFN) treatment (1; 2) and is also significantly induced by viral infection (3) and lipopolysaccharide (LPS) (4). The sequence of the ISG15 protein possesses significant homology to a diubiquitin sequence, and ISG15 can cross-react with some anti-ubiquitin antibodies (3). ISG15 is released by various cell types and can act as a cytokine leading to proliferation of NK cells (5). ISG15 is induced in the uterine endometrium during early pregnancy and may play a role in embryo implantation (6). ISG15 sequences are absent in yeast, nematode (Caenorabditis), plant (Arabidopsis) and insect (Drosophila), indicating that the ISG15-conjugation system is restricted to higher animals with evolved IFN signaling.

ISG15 apparently conjugates to intracellular proteins through an enzymatically catalyzed isopeptide bond formation (7). Dysregulation of ISG15-conjugation has been observed in laboratory animals to cause decreased life expectancy, brain cell injury, and hypersensitivity to interferon, but the ISG15-target protein interactions were undescribed.

Therefore, there is a need for methods to identify ISG15 target proteins. There is also a need for methods to identify compounds that alter the conjugation of ISG15 with target proteins. Furthermore, there is a need for methods to alter the level of ISG15-conjugated protein in a patient or a sample, and methods to diagnose a patient having a malcondition characterized by an altered level of ISG15-conjugated protein. There is also a need for ISG15-target protein conjugates and antibodies that selectively bind to ISG15-protein conjugates. These methods, conjugates, and antibodies are useful to, among other things, diagnose and treat diseases related to dysregulation of ISG15-conjugation.

### SUMMARY OF THE INVENTION

These and other needs are met by the present invention. The present invention is based, in part, upon the discovery of screening assay designed to identify ISG15 target proteins, and the identification of ISG15 target proteins, such as PLCγ1, JAK1, ERK1, ERK2, and Stat1 using such assays. The discovery of these methods and compositions permit the development of methods to, for example, identify compounds that alter the conjugation of ISG15 with target proteins, methods to alter the level of ISG15-conjugated protein in a patient or in a sample, methods to establish a prognosis for a patient based on predicted responsiveness to interferon treatment, methods to diagnose a patient having a malcondition characterized by an altered level of ISG15-conjugated protein, methods to identify additional ISG15-conjugates, and methods to identify antibodies that selectively bind to ISG15-conjugates.

The present invention is directed to a method for screening for the presence of an ISG15-conjugated protein (“ISG15-conjugate”) in a patient or a sample, a method for diagnosing a patient having an altered level of an ISG15-conjugate, a method for determining the responsiveness of a patient to interferon treatment, a method for identifying an antibody that selectively binds to an ISG15-conjugate, a method for promoting and/or enhancing wound healing, a method for increasing the motility of a cell, a method for increasing phagocytotic activity of a cell, and a method for treating a disease characterized by dysregulation of ISG15-conjugation. The present invention is also directed to a method for identifying a target protein that can couple to ISG15 to form an ISG15-conjugate, and a method for identifying an agent that alters the conjugation of ISG15 with a target protein.

In one embodiment of the present invention, screening assays designed to identify a target protein involves contacting a sample suspected of containing an ISG15-conjugate with an antibody that selectively binds to ISG15, or to an ISG15-conjugate, and separating the ISG15-conjugate from the sample. Once separated from the sample, the target protein that is present in the ISG15-conjugate can be identified. Examples of samples that may be used within the methods of the invention include, but are not limited to, cell lysates from tissue culture cells, cells from human blood, and clinical biopsies.

In one example, an ISG15-conjugate can be separated through use of a first antibody that binds to ISG15 alone, to ISG15 when coupled to a target protein, and/or to an ISG15-conjugate. Following separation, the target protein that is present in the ISG15-conjugate can be identified, for example, through the use of one or more antibodies having known binding specificities. In another example, an antibody having a known binding specificity can be contacted with the separated ISG15-conjugate singularly, or in combination with other antibodies having known binding specificities.

In one embodiment, the antibody that binds to ISG15, to ISG15 when coupled to a target protein, and/or an ISG15-conjugate, can be immobilized. In another embodiment, an antibody that binds to ISG15, to ISG15 when coupled to a target protein, and/or to an ISG15-conjugate may be free in solution. In a particular embodiment, the first antibody used to separate the ISG15-conjugate from the sample is removed prior to contacting the ISG15-conjugate with a second antibody that is used to identify the target protein within the ISG15-conjugate. In another particular embodiment, the first antibody used to separate the ISG15-conjugate from the sample is present when a second antibody is contacted with the separated ISG15-conjugate to identify the target protein. The antibody used to separate the ISG15-conjugate from the sample may bind to the ISG15 portion, the target protein portion, and/or a combination of the ISG15 and target protein portion of the ISG15-conjugate. The antibody used to identify the target protein portion of the ISG15-conjugate may bind to the target protein or to a combination of the target protein and ISG15. Numerous antibodies and antibody fragments may be used in accordance with the methods of the present invention. Such antibodies and antibody fragments include, but are not limited to, polyclonal antibodies, monoclonal antibodies, single-chain antibodies, Fab fragments, humanized antibodies, and the like.

The present invention is also directed toward a method for identifying whether a candidate agent alters the conjugation of ISG15 with a target protein. This alteration may be exhibited, for example, through inhibiting, decreasing, increasing or activating such conjugation. The method may be conducted by in vitro or in vivo techniques. In one embodiment, the method generally involves incubating a reaction mixture containing a combination of ISG15, the selected target protein, and the conjugation enzyme UBE1L under conditions designed to enable UBE1L to couple ISG15 to the target protein to form an ISG15-conjugate. After establishing control test results for conjugate formation in the absence of a candidate agent, the candidate agent of concern is added to the reaction mixture and the production of ISG15-conjugate is determined. Comparison of the test results with control results allows a determination whether the candidate agent alters (for example, inhibits, decreased, increased or activates) conjugation. The rate of conjugate formation, amount of conjugate formed, redirection of the enzyme as well as any other reaction parameters and/or results can be used to determine whether the candidate agent alters formation of the ISG15-conjugate. The test can be conducted in vitro, for example, by appropriate combination in aqueous medium. The test can be conducted in vivo, for example, by appropriate incubation in a cellular medium wherein the cells are engineered to express any one or any combination of ISG15, the target protein and the UBE1L enzyme, and the candidate agent exogenously combined.

The present invention is also directed to a composition comprising an isolated ISG15-conjugate. Embodiments of ISG15-conjugates include, without limitation, ISG15-conjugates in which the target protein comprises phospholipase Cγ1, Jak1, ERK1, ERK2, or Stat1. In a particular, non-limiting embodiment, the composition comprises a purified ISG15-conjugate. In another particular, non-limiting embodiment, the composition comprises a substantially purified ISG15-conjugate. In another particular, non-limiting embodiment, the composition comprising a partially purified ISG15-conjugate. In a specific embodiment, the composition comprising an isolated ISG15-conjugate, purified ISG15-conjugate, substantially purified ISG15-conjugate, or partially purified ISG15-conjugate further comprises a pharmaceutically acceptable carrier.

The present invention is also directed to a composition comprising an isolated complex having (1) an ISG15-conjugate and (2) an antibody that selectively binds to the conjugate. In particular, non-limiting embodiments, the compositions comprise ISG15-phospholipase Cγ1, ISG15-Jak1, ISG15, ERK1, ISG15-ERK2, ISG15-Stat1, and/or any combination thereof. In a particular, non-limiting embodiment, the composition comprises a purified complex of an ISG15-conjugate and an antibody that selectively binds to the conjugate. In another particular, non-limiting embodiment, the composition comprises a substantially purified complex of an ISG15-conjugate and an antibody that selectively binds to the conjugate. In another particular, non-limiting embodiment, the composition comprises a partially purified complex of an ISG15-conjugate and an antibody that selectively binds to the conjugate.

The present invention is also directed to a method for altering the level of ISG15-conjugated protein in a patient. In one embodiment, the method comprises administering to the patient an effective amount of an ISG15-conjugate, or variant thereof, that alters the conjugation of ISG15 with a target protein. In another embodiment, the method comprises administering to the patient an effective amount of an agent that alters the conjugation of ISG15 with a target protein. The agent may be identified, for example, according to the methods described herein. An effective amount may be an amount that alters the level of ISG15-conjugated protein in the patient.

The present invention also provides a method for altering the level of ISG15-conjugated protein in a sample. In one embodiment, the method comprises contacting the sample with an effective amount of an ISG15-conjugate, or variant thereof, that alters the conjugation of ISG15 with a target protein. In another embodiment, the method comprises contacting the sample with an effective amount of an agent that alters the conjugation of ISG15 with a protein. The agent may be identified, for example, according to the methods described herein. An effective amount may be an amount that alters the level of ISG15-conjugated protein in the sample.

The present invention also provides a method to diagnose a patient having a malcondition characterized by an altered level of an ISG15-conjugate. In one embodiment, the method comprises contacting an antibody that is substantially selective for an ISG15-conjugate with a sample from a patient suspected of having an altered level of an ISG15-conjugate, and detecting the level of ISG15-conjugate in the sample. An alteration in the level of ISG15-conjugate, as compared with the level of ISG15-conjugate in a sample from a normal patient or patients, indicates an malcondition characterized by an altered level of ISG15-conjugate.

The present invention also provides antibodies that substantially selectively bind to an ISG15-conjugate. In particular, non-limiting embodiments, the antibodies of the present invention selectively bind to an ISG15-phospholipase Cγ1 conjugate, an ISG15-Serpin2a conjugate, an ISG15-JAK1 conjugate, an ISG15-ERK1 conjugate, an ISG15-ERK2 conjugate, and/or an ISG15-Stat1 conjugate. In a particular, nonlimiting, embodiment, the invention provides an antibody that selectively binds to an ISG15-conjugate (comprising ISG15 and a target protein) but does not selectively bind to ISG15 alone and does not selectively bind to the respective target protein alone. Accordingly, such antibodies will be selectively recognize only the ISG15-conjugate, as a complex, and not its respective components when isolated individually.

The present invention also provides a method to increase wound healing comprising contacting a cell with a composition comprising an UBP43 inhibitor or an ISG15-conjugate. In a particular, non-limiting embodiment, the composition further comprises a protein that participates in wound healing.

The present invention also provides a method to increase the motility of a cell comprising contacting the cell with a composition comprising a UBP43 inhibitor or an ISG-conjugate. In a particular, non-limiting embodiment, the composition further comprises a protein that participates in cell motility. Such methods may be used, for example, to promote wound healing and/or to stimulate immune response to a pathogen.

The present invention also provides a method to increase the phagocytotic activity of a cell comprising contacting the cell with a composition comprising an UBP43 inhibitor or an ISG15-conjugate. In a particular, non-limiting embodiment, the composition further comprises a protein that participates in phagocytosis. Such methods may be used, for example, to promote wound healing and to stimulate immune response to a pathogen.

The present invention is also provides a method to modulate conjugation of ISG15 within a patient comprising administering to the patient a composition comprising an ISG-conjugate. In another embodiment, the method comprises administering to the patient a composition comprising an agent capable of modulating ISG15 conjugation identified through use of the methods of the invention.

The present invention also provides a method to treat diseases related to cellular proliferation (for example, cancer), diseases related to interferon dysregulation, bacterial diseases, or viral diseases. In one embodiment, the method comprises administering to a patient in need of such treatment an effective amount of a composition comprising an ISG15 conjugate, or variant thereof. In another embodiment, the method comprises regulating the activity of an ISG15 target protein in a patient. In a further embodiment, the level and/or activity of the target protein is regulated in response to interferon. In a specific embodiment, the method comprises administering to a patient in need of such treatment an agent that increases coupling of a target protein with ISG15. In a further embodiment, the increased coupling prevents, or reduces the ability of, the target protein from causing cell proliferation. In another further embodiment, the increased coupling enables, or enhances the ability of, the target protein to cause cell proliferation.

### BRIEF DESCRIPTION OF FIGURES

FIG. 1 depicts high-throughput western blot identification of ISGylated proteins. IGS15-conjugates purified by immuno-affinity chromatography on anti-ISG15 IgG-resin were screened with 860 monoclonal antibodies at BD Biosciences Transduction Laboratories with PowerBlot. Relevant fragments of templates are shown. (A) Template D lanes 5-7; (B) Template F lanes 38-40; (C) Template E lanes 8-10. Expected positions of indicated proteins (middle lanes) are shown with arrows and observed conjugates are shown with asterisks. Positions of molecular weight markers and heavy (HC) and light (LC) chains of immunoglobulin are shown.

FIG. 2 shows that ISG15-modified PLCγ1, ERK1 and JAK1 are detected in reciprocal immunoprecipitations. (A) Proteins were precipitated with nonspecific isotype control mAbs (N) or with specific anti-ISG15 mAb (S). Ten micrograms of whole cell lysate was loaded (L) to locate positions of unmodified proteins (shown with arrows). Panels were probed with anti-PLCγ1, anti-Jak1, or anti-ERK1 monoclonal antibodies. (B) Proteins were precipitated with nonspecific isotype control mAbs (N) or with specific (S) anti-PLCγ1, anti-JAK1, or anti-ERK1 monoclonal antibodies. Panels were probed with rabbit anti-human ISG15 antibodies (left) and reprobed with the antibodies used for IP (right). Positions of unmodified proteins are shown with arrows and positions of conjugates are shown with asterisks. Positions of molecular weight markers and immunoglobulin heavy chain (HC) are shown on the left.

FIG. 3 shows that PLCγ1 and ERK1 are modified by ISG15 in murine embryonic fibroblasts. ISG15-conjugates were immunoprecipitated from untreated or IFNβ-induced UBP43^{+/+} and IFNβ-induced UBP43^{-/-} murine embryonic fibroblasts (MEFs). Immunocomplexes (lanes 1-3) and cell lysates (lane 4) were subjected to western blot with anti-PLCγ1 and anti-ERK1 mAbs. Positions of unmodified proteins are shown with arrows and positions of conjugates are shown with asterisks. Positions of molecular mass standards and location of IgG heavy chain (HC) are indicated on the left.

FIG. 4 shows that Stat1 is modified by ISG15 in murine and human tissues. (A) Total cell lysates of indicated tissues were resolved on SDS-PAGE and western blotted with anti-Stat1 antibodies. Isolated bone marrow cells were treated in vitro with 100 U/ml of IFNβ for 1 or 24 hours. Thymi were excised from mice injected with poly(I—C) 1 or 24 hours prior to sacrifice. (B) Reciprocal immunoprecipitations from IFNβ treated UBP43^{+/+} and UBP43^{-/-} thymocytes were performed using rabbit anti-Stat1 or anti-ISG15 antibodies and then probed with anti-ISG15 or anti-Stat1, respectively. Proteins were precipitated with nonspecific rabbit IgGs (N) or with specific antibodies (S). Ten micrograms of whole cell lysate was loaded (L) to locate positions of unmodified proteins. (C) ISG15-conjugates immuno-affinity purified on anti-human ISG15 mAb resin from human thymus (same as in FIG. 1) and probed with polyclonal anti-ISG15 or anti-Stat1.

FIG. 5 shows that proteasome inhibitors do not affect the level of ISG15-conjugation. (A) UBP43^{+/+} and UBP43^{-/-} MEFs were incubated with or without IFNβ for 18 hours and were treated with lactacystin at a final concentration of 5 μM for 3 hours. An equal volume of DMSO (vehicle) was added to control samples. Cell lysates (10 μg total protein) were resolved on 8-18% minigel and western blot was performed with rabbit anti-mouse ISG15. The membrane was stripped and reprobed with anti-Ub serum. (B) UBP43^{+/+} (not treated with IFN) and UBP43^{-/-} (incubated with IFNβ for 18 hours) MEFs were treated with MG132 at a final concentration of 10 μM for 3 hours. An equal volume of DMSO (vehicle) was added to control samples. PLCγ1 was immunoprecipitated and immunocomplexes were resolved on 7% minigel. The membrane was probed with anti-ISG15 and, after stripping, was reprobed with anti-PLCγ1. Positions of unmodified PLCγ1 are shown with arrows and positions of conjugates are shown with asterisks. Cell lysates (10 μg total protein) were also resolved on an identical minigel to assess amount of ISG15- and Ub-conjugates. Western blot was performed with rabbit anti-mouse ISG15 and after stripping the membrane was reprobed with anti-Ub serum.

FIG. 6 shows increased migration and phagocytosis in UBP43^{-/-} cells. (A) An equal number of UBP43^{+/+} and UBP43^{-/-} MEFs were seeded on a fibronectin-coated plate and cultured for 24 hours. Migration into the wound is shown 20 hours after the wound was created. (B) Peritoneal macrophages from UBP43^{+/+} and UBP43^{-/-} mice were harvested, plated on a glass chamber and allowed to adhere for 15 minutes at 37° C. Fluorescence from uningested particles was quenched and images were acquired using fluorescence microscopy.

### DEFINITIONS

“Treatment” refers to both therapeutic treatment and prophylactic or preventative measures. Those in need of treatment include those already with the malcondition as well as those in which the malcondition is to be prevented.

A “malcondition” is any condition, such as a disorder or disease, in a mammal that would benefit from treatment with a pharmaceutical composition. Accordingly, a malcondition includes, without limitation, chronic and acute malconditions. A malcondition also includes, without limitation, a pathological condition that predisposes the mammal to a disorder or disease.

An “acceptor molecule” refers to a molecule that is excited by light emitted by a donor molecule. Such molecules are known and are commercially available (Molecular Probes, Inc., Eugene Oreg., 97402).

A “donor molecule” refers to a molecule that is excited by fluorescent light and which emits light that causes excitation of an acceptor molecule. Such molecules are known and are commercially available (Molecular Probes, Inc., Eugene Oreg., 97402).

The terms “protein,” “peptide” and “polypeptide” are used interchangeably herein.

It should be noted that the indefinite articles “a” and “an” and the definite article “the” are used in the present application, as is common in patent applications, to mean one or more unless the context clearly dictates otherwise.

As used herein, the term “protein” includes variants or biologically active fragments of a polypeptide, for example a target protein such as but not limited to phospholipase Cγ1, JAK1, ERK1, ERK2, or Stat1. A “variant” of the protein is a protein that is not completely identical to a native protein (also see definition of “variant” below). A variant protein can be obtained, for example, by altering the amino acid sequence by insertion, deletion or substitution of one or more amino acids according to a conservative amino acid exchange. The amino acid sequence of the protein is modified, for example by substitution, to create a polypeptide having substantially the same or improved qualities as compared to the native polypeptide. A “conservative amino acid exchange” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gin; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cysteine, tyrosine, asparagine and glutamine having uncharged polar side chains; aspartate and glutamate having acidic side chains; and lysine, arginine, and histidine having basic side chains (Stryer, L. Biochemistry (2d edition) W. H. Freeman and Co. San Francisco (1981), p. 14-15; Lehninger, A. Biochemistry (2d ed., 1975), p. 73-75).

“Variant” refers to any pharmaceutically acceptable derivative, analogue, or fragment of a polypeptide (e.g., ISG15-conjugate) described herein. A variant also encompasses one or more components of a multimer, multimers comprising an individual component, multimers comprising multiples of an individual component (e.g., multimers of a reference molecule), a chemical breakdown product, and a biological breakdown product. In particular, non-limiting embodiments, an ISG15 or ISG15-conjugate may be a “variant” relative to a reference ISG15 or ISG15-conjugate by virtue of one or more alterations in amino acid sequence, including without limitation, substitution, deletion or addition of one or more amino acid residues. In another particular, non-limiting embodiment, ISG15 or ISG15-conjugate may be chemically modified without changing the amino acid sequence of the molecule by for example, linking with targeting molecules. Accordingly, chemical modification may be used to create variants of the polypeptides of the invention that have altered charge, solubility, stability, localization, and/or targeting. Also, a variant may include amino acid residues not present in the corresponding native protein, or may include deletions relative to the corresponding native protein. A variant may also be a truncated “fragment” as compared to the corresponding native protein, i.e., only a portion of a full-length protein. Protein variants also include peptides having at least one D-amino acid.

“Conservative amino acid exchange” refers to the exchange for one amino acid for another in a polypeptide chain. Preferred exchanges include, for example; aspartic-glutamic as acidic amino acids; lysine/argininelhistidine as basic amino acids; leucine/isoleucine, methionine/valine, alanine/valine as hydrophobic amino acids; serine/glycine/alanine/threonine as hydrophilic amino acids. Conservative amino acid exchange also includes groupings based on side chains. Members in each group can be exchanged with another. For example, a group of amino acids having aliphatic side chains is glycine, alanine, valine, leucine, and isoleucine. These may be exchanged with one another. A group of amino acids having aliphatic-hydroxyl side chains is serine and threonine. A group of amino acids having amide-containing side chains is asparagine and glutamine. A group of amino acids having aromatic side chains is phenylalanine, tyrosine, and tryptophan. A group of amino acids having basic side chains is lysine, arginine, and histidine. A group of amino acids having sulfur-containing side chains is cysteine and methionine. For example, replacement of a leucine with an isoleucine or valine, an aspartate with a glutamate, a threonine with a serine, or a similar replacement of an amino acid with a structurally related amino acid may be accomplished to produce peptides used in the invention.

The term “ISG15-conjugate” includes polypeptides and peptidomimetics that are bound within the proteolytic site of UBP43. ISG15-conjugates may be bound and cleaved by UBP43, or may be bound but not cleaved by UBP43. An ISG15-conjugate typically includes an ISG15 portion, and a coupled partner portion. Coupled partners include, but are not limited to, phospholipase Cγ1, Jak1, ERK1, and Stat1. Accordingly, ISG15-conjugates include ISG15-phospholipase Cγ1, ISG15-Jak1, ISG15-ERK1, and ISG15-Stat1. ISG15-conjugates can also include fragments of: ISG15, a coupled partner, ISG15-phospholipase Cγ1, ISG15-Jak1, ISG15-ERK1, and ISG15-Stat1 that are bound within the proteolytic site of UBP43. In addition, ISG15-conjugates include polypeptide segments that are bound within the proteolytic site of UBP43 and cleaved. These ISG15-conjugates may include bonds that allow them to be bound by UBP43, but not proteolytically cleaved by UBP43.

The term “peptidomimetic” or “peptide mimetic” describes a peptide analog, such as those commonly used in the pharmaceutical industry as non-peptide drugs, with properties analogous to those of the template peptide. (Fauchere, J., Adv. Drug Res., 15: 29 (1986) and Evans et al., J. Med. Chem., 30: 1229 (1987)). Generally, peptidomimetics are structurally similar to a paradigm polypeptide (i.e., a polypeptide that has a biochemical property or pharmacological activity), but have one or more peptide linkages optionally replaced by a linkage such as, —CH2NH—, —CH2S—, —CH2—CH2—, —CH═CH—(cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data, 1: 3 (1983); Morley, J. S., Trends. Pharm. Sci., pp. 463-468 (1980); Hudson, D. et al., Int. J. Pept. Prot. Res., 14: 177-185 (1979); Spatola et al., Life Sci., 38: 1243 (1986); Harm, J. Chem. Soc. Perkini Trans I, 307-314 (1982); Almquist et al., J. Med. Chem., 23: 1392 (1980); Jennings-White et al., Tetrahedron Lett., 23: 2533 (1982); Szelke et al., European Appln. EP 45665 (1982) CA: 97: 39405 (1982); Holladay et al., Tetrahedron Lett., 24: 4401 (1983); and Hruby, Life Sci., 31: 189 (1982). Advantages of peptide mimetics over natural polypeptide embodiments may include more economical production, greater chemical stability, altered specificity, reduced antigenicity, and enhanced pharmacological properties such as half-life, absorption, potency and efficacy. Substitution of one or more amino acids within polypeptide or peptide mimetic with a D-amino acid of the same type (e.g., D-lysine in place of L-lysine) may be used to generate polypeptides and peptide mimetics that are more stable and more resistant to endogenous proteases.

The term “antibody” is used in the broadest sense, and covers monoclonal antibodies (including full length monoclonal antibodies), polyclonal antibodies, multispecific antibodies (e.g., bispecific antibodies), and antibody fragments (e.g., Fab, F(ab′)₂, Fd and Fv) so long as they exhibit antigen binding.

The term “antibody fragment” refers to a portion of a full-length antibody, generally the antigen binding or variable region. Examples of antibody fragments include Fab, Fab′, F(ab′)₂, Fd and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of crosslinking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ and Fd fragments.

The term “monoclonal antibody” as used herein refers to an antibody obtained from a population of substantially homogeneous antibodies, i.e., the individual antibodies composed of the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by a hybridoma or phage infected bacterial culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, Nature 256:495 (1975), or maybe made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

The term “diabodies” refers to a small antibody fragments with two antigen-binding sites, which fragments include a heavy chain variable region (V_{H}) connected to a light chain variable domain (V_{L}) in the same immunopolypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Holliger et al., Proc. Natl. Acad. Sci. USA 90: 6444-6448 (1993).

A “pharmaceutical composition” includes an ISG15-conjugate or agent identified according to the invention, for example ISG15-Stat1, in combination with a pharmaceutically acceptable carrier. The ISG15-conjugates or agents of the invention may be formulated for oral or parenteral administration (e.g., by injection, for example, bolus injection or continuous infusion) and may be presented in unit dosage form in ampules, prefilled syringes, small volume infusion containers or multi-dose containers with an added preservative. The ISG15-conjugates or agents of the invention may take such forms as liposomes and may contain formulatory agents such as suspending, stabilizing and/or dispersing agents.

For topical administration to the epidermis, the ISG15-conjugates or agents of the invention may be formulated as ointments, creams or lotions, or as the active ingredient of a transdermal patch. Suitable transdermal delivery systems have been disclosed (U.S. Pat. Nos. 4,788,603; 4,931,279; 4,668,506; and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The compounds can also be delivered via iontophoresis (U.S. Pat. Nos. 4,140,122; 4,383,529; and 4,051,842).

Pharmaceutical compositions suitable for topical administration in the mouth include unit dosage forms such as lozenges comprising one or more ISG15-conjugates or agents of the invention in a flavored base, usually sucrose and acadia or tragacanth; pastilles comprising the ISG15-conjugates or agents of the invention in an inert base such as gelatin and glycerin or sucrose and acacia; mucoadherent gels, and mouthwashes comprising the ISG15-conjugates or agents of the invention in a suitable liquid carrier.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the ISG15-conjugates or agents of the invention with the softened or melted carriers) followed by chilling and shaping in molds. Typically the conjugates and agents are contained within liposomes.

Pharmaceutical compositions suitable for vaginal administration may be presented as pessaries, tampons, creams, gels, pastes, foams or sprays containing the liposome containing ISG15-conjugates or agents, and such carriers are well known in the art.

For administration by inhalation, liposomes containing ISG15-conjugates or agents according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For intra-nasal administration, liposomes containing the ISG15-conjugates or agents of the invention may be administered via a liquid spray, such as via a plastic bottle atomizer. Typical of these are the Mistometer® (isoproterenol inhaler—Wintrop) and the Medihaler® (isoproterenol inhaler—Riker).

For topical administration to the eye, liposomes containing the ISG15-conjugates or agents can be administered as drops, gels (U.S. Pat. No. 4,255,415), gums (U.S. Pat. No. 4,136,177) or via a prolonged-release ocular insert (U.S. Pat. Nos. 3,867,519 and 3,870,791).

The amount of the ISG15-conjugates, agents, or combinations thereof that are administered and the frequency of administration to a given human patient will depend upon a variety of variables related to the patient's psychological profile and physical condition. For evaluations of these factors, see J. F. Brien et al., Eur. J. Clin. Pharmacol., 14, 133 (1978); and Physicians' Desk Reference, Charles E. Baker, Jr., Pub., Medical Economics Co., Oradell, N.J. (41st ed., 1987).

“Pharmaceutically acceptable salts” of the ISG15-conjugates or agents of the invention include, but are not limited to, the nontoxic addition salts with organic and inorganic acids, such as the citrates, bicarbonates, malonates, tartrates, gluconates, hydrochlorides, sulfates, phosphates, and the like.

“Polypeptides” and “Proteins” are used interchangeably herein. Polypeptides and proteins can be expressed in vivo through use of prokaryotic or eukaryotic expression systems. Many such expressions systems are known in the art and are commercially available. (Clontech, Palo Alto, Calif.; Stratagene, La Jolla, Calif.). Examples of such systems include, but are not limited to, the T7-expression system in prokaryotes and the bacculovirus expression system in eukaryotes. Polypeptides can also be synthesized in vitro, e.g., by the solid phase peptide synthetic method or by in vitro transcription/translation systems. The synthesis products may be fusion polypeptides, i.e., the polypeptide comprises the polypeptide variant or derivative according to the invention and another peptide or polypeptide, e.g., a His, HA or EE tag. Such methods are described, for example, in U.S. Pat. Nos. 5,595,887; 5,116,750; 5,168,049 and 5,053,133; Olson et al., Peptides, 9, 301, 307 (1988). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). These polypeptides can be further purified by fractionation on immunoaffinity or ion-exchange columns; ethanol precipitation; reverse phase HPLC; chromatography on silica or on an ion-exchange resin such as DEAE; chromatofocusing; SDS-P AGE; ammonium sulfate precipitation; gel filtration using, for example, Sephadex G-75; or ligand affinity chromatography.

The polypeptides include the exchange of at least one amino acid residue in the polypeptide for another amino acid residue, including exchanges that utilize the D rather than L form, as well as other well known amino acid analogs, e.g., N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, N-methyl-alanine, para-benzoyl-phenylalanine, phenyl glycine, propargylglycine, sarcosine, N-acetylserine, N-formylmethionine, 3-methylhistidine, 5-hydroxylysine, and other similar amino acids and imino acids and tert-butylglycine.

The term “UBP43” refers to a polypeptide that exhibits ISG15-specific proteolytic activity. This activity is able to cleave ISG15-conjugates. The amino acid sequence of a human UBP43 has accession number NP_{—}059110, and the nucleic acid sequence has accession GeneBank accession number NM_{—}017414. The amino acid sequence of a mouse UBP43 has accession number NP_{—}036039, and the nucleic acid sequence has accession GeneBank accession number NM_{—}011909. Thus, polypeptides having amino acid deletions, additions, conservative exchanges, or combinations thereof are included within the definition of UBP43 as long as they retain the ISG15-conjugate cleavage activity exhibited by UBP43 polypeptides described by accession numbers NP_{—}059110 or NP_{—}036039.

A “substrate” refers to a polypeptide or peptidomimetic that is bound within the proteolytic site of UBP43 and which produces a detectable product when cleaved by UBP43. Examples of substrates include, but are not limited to, ISG15-phospholipase Cγ1, ISG15-Jak1, ISG15-ERK1, and ISG15-Stat1 in which the ISG15 portion of the conjugate is labeled with a first fluorescent dye and the coupled partner portion (for example, phospholipase Cγ1, Jak1, ERK1, or Stat1) is labeled with a second fluorescent dye that can undergo fluorescence resonance energy transfer (FRET) with the first fluorescent dye. Thus, cleavage of the substrate separates the first and second fluorescent dyes and causes a detectable decrease in FRET. Other examples of labels and techniques that may be used to prepare substrates and detect substrate cleavage include, but are not limited to, fluorescent quenching, spin coupling, enzyme-linked immunosorbant assay (ELISA), radio-immunoassay (RIA), and the like.

### DETAILED DESCRIPTION OF THE INVENTION

The present invention is based, in part, upon the discovery that ISG15 is a ubiquitin-like protein, which conjugates to proteins in cells, whereupon the cells undergo significant physiologic change. In a non-limiting embodiment of the invention, the conjugation is an enzymatic process catalyzed by the UBE1L enzyme and forms an isopeptide bond between side chain amine and carboxyl groups of ISG15 and the target protein. In another embodiment, the conjugation is regulated by the UBP43 enzyme that can hydrolyze the linkage through which ISG15 is connected to the target protein.

The extracellular messenger causing the ISG15 cascade is interferon or lipopolysaccharide. The ISG15 cascade is tightly controlled. If conjugation of ISG15 with its target proteins (ISGylation) is not controlled but instead is deregulated so as to produce higher than normal quantities of conjugate, decreased life expectancy, brain cell injury, and hypersensitivity to interferon result. However, if conjugation of ISG15 with its target protein is inhibited, then cellular proliferation occurs. This cellular proliferation leads to disease related to the cellular proliferation, such as cancer. An example of such a cancer is leukemia.

Identification of ISG15 Target Proteins

According to the invention, several ISG15 target proteins have been identified. These include PLCγ1, JAK1, ERK1, ERK2, and Stat1. These identified target proteins serve as sites of action for compounds that affect ISG15 conjugation, as models for identification of other target proteins, and as indicators for diagnosing and treating patients having a malcondition characterized by an alteration in the level of ISG15-conjugation. Such a malcondition can be exemplified by hypersensitivity to interferon treatment.

Accordingly, the present invention provides a composition comprising an isolated ISG15-conjugate. Embodiments of ISG15-conjugates include, without limitation, ISG15-conjugates in which the target protein comprises phospholipase Cγ1, Jak1, ERK1, ERK2, or Stat1. In a particular, non-limiting embodiment, the composition comprises a purified ISG15-conjugate. In another particular, non-limiting embodiment, the composition comprises a substantially purified ISG15-conjugate. In another particular, non-limiting embodiment, the composition comprising a partially purified ISG15-conjugate. In a specific embodiment, the composition comprising an isolated ISG15-conjugate, purified ISG15-conjugate, substantially purified ISG15-conjugate, or partially purified ISG15-conjugate further comprises a pharmaceutically acceptable carrier.

The present invention also provides a composition comprising an isolated complex comprising an ISG15-conjugate and an antibody that selectively binds to the conjugate. In particular, non-limiting embodiments, the compositions comprise ISG15-phospholipase Cγ1, ISG15-Jak1, ISG15-ERK1, ISG15-ERK2, ISG15-Stat1, and/or any combination thereof. In a particular, non-limiting embodiment, the composition comprises a purified complex of an ISG15-conjugate and an antibody that selectively binds to the conjugate. In another particular, non-limiting embodiment, the composition comprises a substantially purified complex of an ISG15-conjugate and an antibody that selectively binds to the conjugate. In another particular, non-limiting embodiment, the composition comprises a partially purified complex of an ISG15-conjugate and an antibody that selectively binds to the conjugate.

Three of the target proteins, PLCγ1, JAK1, and ERK1 are regulators of signal transduction. The transcription factor Stat1, an immediate substrate of JAK1 kinase, was also discovered to be a target protein through use of the methods of the invention. It has been discovered that ISG15-conjugates do not accumulate in cells treated with specific inhibitors of proteasomes. Thus, although it is not to be regarded as a limitation, it is believed that ISG15 is involved in the regulation of multiple signal transduction pathways as opposed to strictly regulating protein degradation. The invention disclosed herein, among other uses, offers models to further elucidate the biochemical function of ISGylation.

Several key regulators of signal transduction (PLCγ1, JAK1, Stat1, ERK1, and ERK2) are coupled to ISG15 to form ISG15-conjugates. Methodology for identification of new ISG15 targets is also a feature of the invention. This methodology enables the study of the function of ISG15 conjugation and elucidates a biological role for ISG15-modification in the regulation of multiple signal transduction pathways.

All of the target proteins identified through use of the methods of the invention are known to be active during signal transduction. The phospholipase C (PLC) isozymes hydrolyze phosphatidyl inositol biphosphate to inositol triphosphate and diacylglycerol (29). The former causes release of calcium from the endoplasmic reticulum, while the latter is an activator of Protein Kinase C (30). PLCγ1 is essential for growth factor-induced cell motility and mitogenesis (31). PLCγ1 knockout mice exhibit retarded embryonic growth and lethality in midgestation (32), illustrating its importance in normal growth and development. Overexpression of PLCγ1 is evident in several forms of cancer and it has been identified as a key mediator of PDGF-dependent cellular transformation (33).

The significance of the identification of these target proteins is shown by their participation in an INF initiated signal cascade. The families of signal cascade proteins represented by the target proteins (e.g., the Jak family and the Erk family) have control roles in cellular growth, differentiation and death.

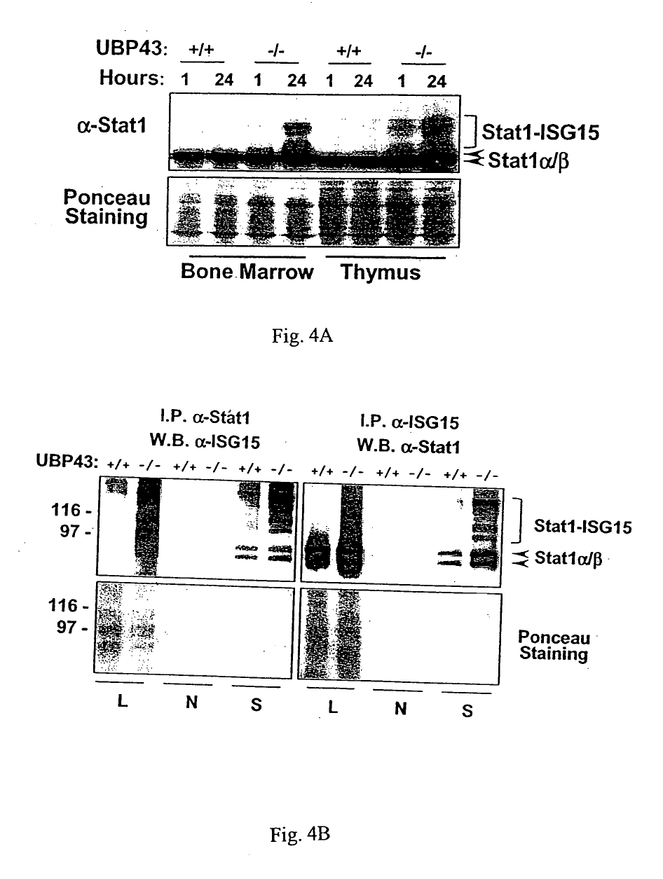

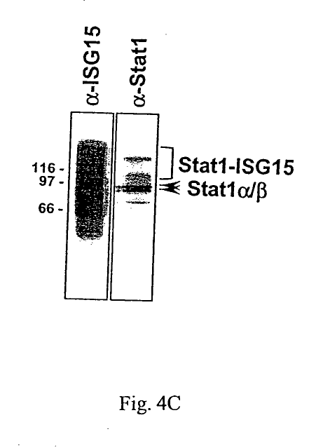

For example, the Jak family of receptor-associated protein kinases (JAK1, 2, 3 and Tyk2) are directly involved in response to interferon (“IFN”) and other cytokines (34). JAK1 is rapidly phosphorylated in response to IFN and is required for the phosphorylation of the transcription factor Stat1 (25). As disclosed herein, Stat1 is also modified by ISG15 in poly(I—C) or IFNβ treated murine cells, and Stat1 is also modified in human thymus (FIG. 4C). JAK1-deficient mice exhibit perinatal lethality, apparently because of defective neural function, and defective lymphoid development (35). Stat1 knockout mice are viable and display no developmental effects, however, all the physiological functions associated with the IFNs are absent, leading to a remarkable sensitivity to viral infections and other pathological agents (36; 37).

In another example, the family of kinases known as ERKs or MAPKs are activated after cell stimulation by a variety of hormones and growth factors. Cell stimulation induces a signaling cascade that leads to phosphorylation of MEK (MAPK/ERK kinase) which, in turn, activates ERK. Numerous proteins represent the downstream effectors for the active ERK and indicate that ERK acts during control of cell proliferation, differentiation, as well as regulation of the cytoskeleton and migration (38). Elevated ERK activity is associated with some cancers. Thus several members of this signaling cascade have been considered important drug targets in therapies of cancers and inflammatory diseases (39).

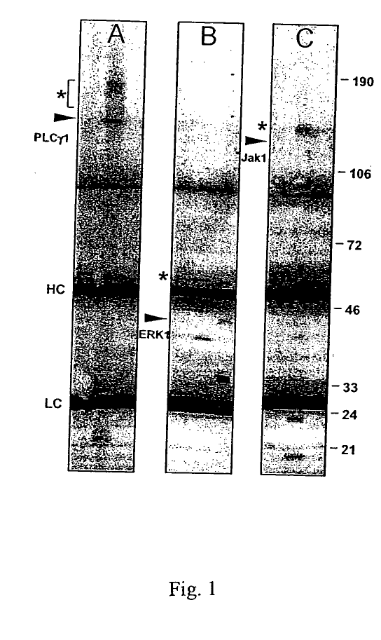

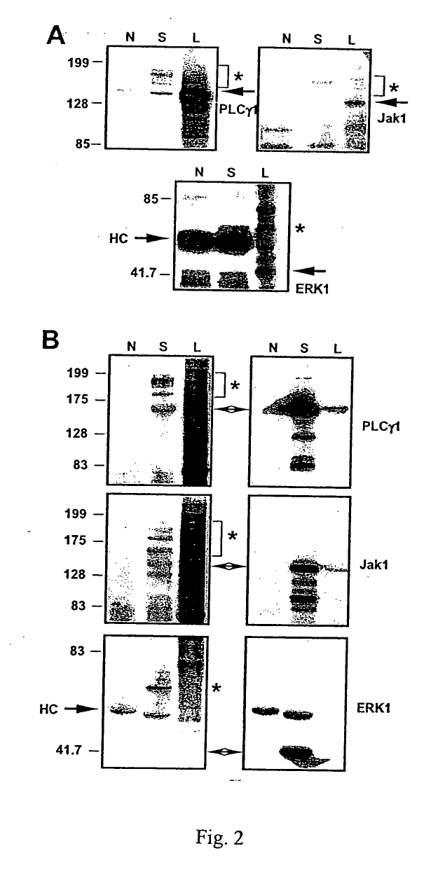

The target proteins identified (ERK1, ERK2, PLCγ1, Jak1, and Stat1) have diverse biochemical functions. Without being bound by any particular theory, it is believed that ISG15-conjugation alters basic characteristics of the protein, e.g., charge, solubility, stability, localization etc., rather than modifies a specific biological activity (e.g. enzymatic or DNA-binding activity) of a given target. In most of the examples disclosed herein, fragments of PLCγ1, JAK1, ERK1, ERK2, (FIGS. 1 and 2B) as well as Stat1 were observed. Thus, ISG15-conjugation may promote degradation of targeted proteins via a pathway alternative to proteasomal degradation.

Coordinated induction of ISG15, UBP43 and UBE1L indicates that ISG15-conjugation is a dynamic and highly controlled process. In fact, the ISG15-coupling enzyme, UBE1L, was found to be absent in 14 different lung cancer cell lines. Therefore, a decrease of ISG15-conjugation is thought to contribute to carcinogenesis (40). Certain viruses can specifically block conjugation or synthesis of ISG15 (11), possibly in attempt to suppress host-cell suicide and inflammatory response. Dysregulation of ISG15-conjugation due to deletion of UBP43 leads to decreased life-expectancy, brain cell injury, and hypersensitivity to interferon. The participation of ISG15-conjugation in control of cellular growth regulation is also suggested by the action of IFN to suppress cellular proliferation.

In one embodiment of the present invention, ISG15-conjugation is directly involved in the regulation of several signal transduction pathways in organisms challenged with IFN elicitors. Upon IFN stimulation, the cell fine-tunes the degree of response via ISGylation of Jak1 and Stat1, which are crucial players in the IFN pathway. Moreover, other signaling pathways are also directly affected by means of ISG15-conjugation of respective critical regulators (e.g., PLCγ1, ERK1) in order to orchestrate overall cellular growth, differentiation, and survival.

The target proteins (such as a phospholipase Cγ1, Jak1, ERK1, or Stat1 protein) identified according to the present invention may be substantially identical to the corresponding native protein, or may have about 1%, 1 to 5%, or 5-10% conservative amino acid changes compared to the native amino acid sequence. The definition of a conservative amino acid exchange is provided above.

Preparation of Antibodies

Antibodies of the invention can be prepared using standard techniques. To prepare polyclonal antibodies or “antisera,” an animal is inoculated with an antigen, i.e., a purified immunogenic ISG15-conjugate, and immunoglobulins are recovered from a fluid, such as blood serum, that contains the immunoglobulins, after the animal has had an immune response. For inoculation, the antigen may be bound to a carrier peptide and emulsified using a biologically suitable emulsifying agent, such as Freund's incomplete adjuvant. A variety of mammalian or avian host organisms may be used to prepare antibodies.

Following immunization, Ig is purified from the immunized bird or mammal, e.g., goat, rabbit, mouse, rat, or donkey and the like. For certain applications, particularly certain pharmaceutical applications, it is preferable to obtain a composition in which the antibodies are essentially free of antibodies that do not react with the immunogen. This composition is composed virtually entirely of the high titer, monospecific, purified polyclonal antibodies to the immunogen. Antibodies can be purified by affinity chromatography. Purification of antibodies by affinity chromatography is generally known to those skilled in the art (see, for example, U.S. Pat. No. 4,533,630). Briefly, the purified antibody is contacted with the purified immunogen bound to a solid support for a sufficient time and under appropriate conditions for the antibody to bind to the immunogen. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

In some embodiments of the invention, antibodies are used that bind to ISG15-conjugates, but do not bind to ISG15 or to the target protein of the ISG15-conjugate. These antibodies can be initially isolated through binding the antibody to the ISG15-conjugate. The initial antibody preparation is then contacted with ISG15 and the target protein alone to remove antibodies that bind to ISG15 or the target protein. The antibodies resulting from the selection process bind specifically to the ISG15-conjugate. Antibodies that bind specifically to ISG15-conjugates may be used to isolate ISG15-conjugates that include unknown target proteins. Such ISG15-conjugate-specific antibodies may bind to a region of the ISG15-conjugate that is highly conserved among ISG15-conjugates that include a variety of target proteins.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically.

Thus, it will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

Immunologically active fragments of an antibody that binds ISG15, ISG15 when coupled to a target protein, and/or ISG15-conjugate are also within the scope of the present invention, e.g., the F(ab) fragment and scFv antibodies, as are partially humanized monoclonal antibodies.

The antibodies of the invention may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the invention. Such solid supports include permeable and semipermeable membranes, glass beads, plastic beads, latex beads, plastic microtiter wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable region (V_{H}) followed by a number of constant regions. Each light chain has a variable region at one end (V_{L} and a constant region at its other end. The constant region of the light chain is aligned with the first constant region of the heavy chain, and the light chain variable region is aligned with the variable region of the heavy chain. The variable region of either chain has a triplet of hypervariable or complementarity determining regions (CDR's) spaced within a framework sequence as explained below. The framework and constant regions of the antibody have highly conserved amino acid sequences such that a species consensus sequence may typically be available for the framework and constant regions. Particular amino acid residues are believed to form an interface between the light and heavy chain variable regions (Chothia et al., J. Mol. Biol. 186:651-63, 1985; Novotny and Haber, Proc. Natl. Acad. Sci. USA 82:4592-4596, 1985).

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable region interact to define an antigen binding site on the surface of the V_{H}-V_{L} dimer. Collectively, the six CDRs confer antigen binding specificity to the antibody. However, even a single variable region (or half of an Fv including only three CDRs specific for an antigen) has the ability to recognize and bind antigen, although at a lower affinity than the entire binding site.

The Fab fragment [also designated as F(ab)] also contains the constant region of the light chain and the first constant region (CHI) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant regions have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art. The light chains of antibodies (immunoglobulin) from any vertebrate species can be assigned to one of two clearly distinct types, called kappa (κ) and lambda (λ), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these maybe further divided into subclasses (isotypes), e.g., IgG-1, IgG-2, IgG-3 and IgG4; IgA-1 and IgA-2. The heavy chains constant domains that correspond to the different classes of immunoglobulins are referred to as α, δ, ε, γand μ, respectively. The subunit structures and three-dimensional configurations of different classes of immunoglobulins are well known. The preferred immunoglobulin for use with the present invention is immunoglobulin IgG.

Monoclonal antibodies, fragments and single chains thereof include “chimeric” forms in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81:6851-6855 (1984).

Antibodies also include fully human forms in which the entire sequence is derived from human immunoglobulins (recipient antibody) including the complementary determining region (CDR) of the immunopolypeptide. In some instances, Fv framework residues of the human immunoglobulin are replaced by corresponding non-human residues. Furthermore, an immunopolypeptide include residues which are found neither in a human immunoglobulin nor in a non-human mammalian sequence.

“Single-chain Fv” or “sFv” antibody fragments include the V_{H} and V_{L} regions of an antibody, wherein these regions are present in a single immunopolypeptide chain. Generally, the Fᵥ immunopolypeptide further includes an immunopolypeptide linker between the V_{H} and V_{L} regions which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The antibodies of the invention are useful for detecting or determining whether a compound affects the level or amount of ISG15-conjugate. The antibodies are contacted with a mammalian sample, e.g., tissue biopsy, mammalian physiological fluid including cells, cultured cells, nuclei, or extracts thereof, which have been incubated with a compound for a period of time and under conditions sufficient for antibodies to bind to the conjugate, so as to form a binary complex between at least a portion of said antibodies and the conjugate. Such times, conditions and reaction media can be readily determined by persons skilled in the art. Then it is determined whether the compound increases or decreases the level or amount of ISG15-conjugate relative to a sample, cells, nuclei or extract not contacted with the compound.

Methods to Identify a Target Protein Conjugated to ISG15 to form an ISG15-Conjugate

The present invention provides methods to identify a target protein that binds ISG15 to form an ISG15-conjugate. Generally, the methods involve separating an ISG15-conjugate from a sample and then determining the identity of the target protein that is coupled to ISG15.

An ISG15-conjugate can be separated from a sample by contacting the sample with an antibody such that a complex is formed which contains the antibody bound to the ISG15-conjugate, and then separating the complex. Any antibody can be used within the methods of the invention that specifically binds to ISG15, or to an ISG15-conjugate, and allows the complex to be separated. In addition, any technique known in the art may be used to isolate a complex containing an antibody bound to an ISG15-conjugate that allows the target protein of the ISG15-conjugate to be identified following separation.

Antibodies that specifically bind to ISG15 are known and have been described (Padovan et al., Cancer Res., 62: 3453 (2002); Malakhova et al., Biol. Chem., 277: 14703 (2002); D'Cunha et al., J. Immunol., 157: 4100 (1996)). In addition, methods to prepare polyclonal and monoclonal antibodies are well known (Harlow et al., Antibodies: A Laboratory Manual (Cold Spring Harbor Pub. 1988)). Examples of types of antibodies that can be used include monoclonal antibodies, polyclonal antibodies, antibody fragments such as Fab fragments, single chain antibodies, humanized antibodies, recombinant antibodies, and the like.

In one embodiment, an antibody is bound to a support and then used to bind and isolate ISG15-conjugates. For example, an antibody may be coupled to a bead that is packed into a liquid chromatography column. A sample may be applied to the column wherein ISG15-conjugates contained within the sample are bound by the immobilized ISG15-specific antibodies. The column can be washed to eliminate unbound materials and then the ISG15-conjugates can be eluted. These ISG15-conjugates can then be used as antigens for the preparation of additional antibodies against the specific ISG15-conjugates. The eluted ISG15-conjugates can also be used in the identification methods described herein below to identify the target protein included in the separated ISG15-conjugate. In another example, an antibody may be coupled to a surface, such as a 96 well tray, to which a sample is applied. The surface can be washed to eliminate unbound material and then the separated ISG15-conjugates can be washed from the surface. Alternatively, the ISG15-conjugates can be left bound to the surface and method described herein below can be used to identify a target protein included in the separated ISG15-conjugate.

In one embodiment, an antibody is contacted with a sample while in solution. In such a case, an antibody that forms a complex with an ISG15-conjugate will include a modification which allows the complex to be separated. For example, an antibody may be coupled to a magnetic bead. Thus, following complex formation between the antibody and the ISG15-conjugate, magnetism may be used to separate the antibody complex from the sample. In another example, an antibody may be coupled to another protein, such as streptavidin, that binds to a second protein, such as biotin, such that a complex containing the antibody can be separated using the second protein. Numerous methods are known in the art to prepare modified antibodies and use them for separation of complexes formed by binding of the modified antibody to form a complex.

In one embodiment, identification of a target protein included within an ISG15-conjugate is accomplished through use of antibodies having known specificities. Such antibodies may be contacted with a separated ISG15-conjugate. If the antibody having a known specificity binds to the ISG15-conjugate, then the identity of the target protein is indicated. A separated ISG15-conjugate can be tested for the presence of a protein for which a specific antibody exists through use of numerous techniques. Examples of such techniques include, but are not limited to, radioimmunoassay (RIA), radioallergosorbent test (RAST), radioimmunosorbent test (RIST), immunradiometric assay (IRMA), fluorescence immunoassay (FIA), sandwich assay, enzyme linked immunosorbent assay (ELISA) assay, and the like.

In one embodiment target protein that is included within an ISG15-conjugate is identified through use of instrumental methods known in the art. For example, an ISG15-conjugate may be isolated as described above or through use of other methods, such as liquid chromatography, and then subjected to analysis by mass spectrometry. Mass spectrometers and methods for their use in determining the identity of proteins have been described (U.S. Pat. Nos. 6,462,337; 6,423,965; 6,322,970). Additionally, the ISG15-conjugate may by treated to cleave ISG15 from the target protein. The target protein can then be separated from ISG15 and the identity of the free target protein may be determined through use of mass spectrometry.

As described herein, many different types of samples may be tested for the presence of an ISG15-conjugate. In one example, a sample can be obtained from cells that have been treated with retinoic acid. Retinoic acid is known to induce the enzyme UBE1L (ubiquitin-activating enzyme E1-like) which acts to couple a target protein to ISG15. Accordingly, treatment of cells with retinoic acid is expected to enrich for ISG15-conjugates in the cell. In another example, a sample may be tissue obtained from a thymus.

Methods to Determine if a Candidate Agent Alters Conjugation of ISG15 with a Target Protein to form an ISG15-Conjugate

The invention provides methods to determine if a candidate agent alters (for example, activates, stimulates, decreases, or inhibits) the coupling of ISG15 to a target protein to form an ISG15-conjugate. Generally, the methods are based on conducting assays in the presence and absence of a candidate agent, and determining if the concentration of one or more ISG15-conjugates is altered within the assay due to the presence of the candidate agent. In one embodiment, these methods can be used to identify candidate agents that act on UBE1L and which are thought to be useful for treating diseases that involve cellular proliferation. Acute promyelocytic leukemia is an example of a disease related to cellular proliferation that is thought to be treatable by agents acting on UBE1L (Kitareewan et al., Proc. Natl. Acad. Sci., 99: 3806 (2002)).

Cell-Based Assays

In one example, the method provides a cell-based method to determine if a candidate agent increases ISG15-conjugate formation within the cell.

The method involves contacting a test cell with a candidate agent and then determining if the ISG15-conjugate concentration within the test cell is altered when compared to the ISG15-conjugate concentration in a control cell that was not contacted with the candidate agent. The concentration of one or more ISG15-conjugates may be determined. Examples of ISG15-conjugates that may be assayed include, but not limited to, ISG15-phospholipase Cγ1, ISG15-JAK1, ISG15-ERK1, ISG15-Stat1, Serpin2a, and ISG15-conjugates identified according to the methods of the invention. The ISG15-conjugate concentration can be determined through use of immunologically based methods with antibodies that recognize the ISG15-conjugates. Antibodies that recognize an ISG15-conjugate, but not ISG15 or the target protein of the ISG15-conjugate are desirable, but not necessary for determining the concentration of specific ISG15-conjugates.

Such methods may be adapted for high-throughput using methods known in the art, e.g., in the pharmaceutical industry. For example, cells may be plated in each of the wells of a 96 well plate. Control cells will not be contacted with a candidate agent. Test cells will be contacted with a candidate agent. Accordingly, numerous different candidate agents may be tested on a single plate. The cells can be incubated with the candidate agent for a given time and then lysed. An aliquot from each well can then be collected and the concentration of the ISG15-conjugate concentration in the aliquot can be determined and compared to a control.

In Vitro Assays

In another example, the invention provides in vitro methods to determine if a candidate agent increases or decreases the ability of UBE1L to couple ISG15 to a target protein. These assays are generally based on incubating UBE1L with ISG15 and a target protein in the presence and absence of a candidate agent, under conditions wherein the UBE1L can couple the ISG15 to the target protein. As described above, coupling of ISG15 to a target protein can be determined through use of immunological methods using antibodies against the ISG15-conjugate. In addition, coupling of ISG15 to a target protein can be determined through use of polyacrylamide gel electrophoresis, and coupling will produce an ISG15-conjugate of higher molecular weight.

The invention also provides a high-throughput assay to identify activators or inhibitors of UBE1L mediated coupling. Generally these methods are based on detecting coupling of ISG15 to a target protein. Generally, ISG15 and target proteins may be used that each have attached thereon a label molecule. Upon coupling of ISG15 to a target protein, a signal may be detected to indicate coupling. Examples of such label molecules include, but are not limited to, molecules used for fluorescence resonance energy transfer (FRET), fluorescent quenching, spin labels, and the like.

For example, fluorescence resonance energy transfer (FRET) may be used to detect coupling of ISG15 to a target protein. Fluorescence energy transfer is a distance-dependent interaction between the electronic excited states of two dye molecules in which excitation is transferred from a donor molecule to an acceptor molecule. The efficiency of FRET is dependent on the inverse sixth power of the intermolecular separation, making it useful over distances comparable with the dimensions of biological macromolecules. Typically, a donor and an acceptor molecule are within from 10-100 angstroms of each other. Typical examples of donor and acceptor molecule pairs include: fluorescein-tetramethylrhodamine, IAEDANS-fluorescein, EDANS-Dabcyl, fluorescein-fluorrescein, BODIPY FL-BODIPY FL, fluroescein-QSY 7 dye, and fluroescein-QSY 9 dye. Dyes and instructions for their use are commercially available and known in the art (Molecular Probes, Inc., Eugene Oreg., 97402). Accordingly, ISG15 can be prepared having a donor molecule and a target protein can be prepared having an acceptor molecule attached such that coupling by UBE1L will locate the donor molecule in close proximity to the acceptor molecule. This close proximity will cause a detectable increase in FRET that can be monitored to determine the activity of UBE1L. Any of the target proteins described herein or identified according to the invention may be used. Protein can be labeled for FRET analysis through use of methods known in the art (Sambrook et al., Molecular Cloning: A Laboratory Manual, 3rd edition, Cold Spring Harbor Press, Cold Spring Harbor, N.Y. (2001)). These methods can be used to determine if a candidate agent increases or decreases the activity of UBE1L. These methods may be adapted for use in automated systems for high-throughput screening of candidate agents. These automated methods are known in the pharmaceutical industry. For example, robotic arms may be used to transfer labeled ISG15, labeled target protein and UBE1L into a well of a 96 well plate. Following incubation, fluorescence resonance energy transfer within the wells of the plate may be detected through use of a plate reader to identify candidate agents that increase or decrease UBE1L activity. Numerous automated systems may be constructed to measure the activity of UBE1L through use of fluorescence resonance energy transfer based methods.

Fluorescent quenching may also be used to determine whether a candidate agent increases or decreases UBE1L activity. Generally, these methods use a labeled ISG15 having attached thereon a fluorescent molecule and a target protein having attached thereon a fluorescent quenching molecule, or the opposite thereof. Upon coupling of ISG15 to a target protein, fluorescence will be quenched. Accordingly, the ability of a candidate agent to increase or decrease the activity of UBE1L can be determined by detecting whether the presence of the candidate agent causes an increase or decrease in fluorescence when compared to a control that lacks the candidate agent. These methods may be readily adapted for use within automated systems. For example, a robotic arm can be used to dispense a labeled ISG15, a labeled target protein, and UBE1L into a control well of a 96 well plate; and a labeled ISG15, a labeled target protein, UBE1L, and candidate agents into the remaining test wells of the 96 well plate. The plate can be incubated and then a fluorescent plate reader can be used to determine the fluorescence in the control well and the fluorescence in the test wells. The level of fluorescence in a test well can be compared to fluorescence in the control well to determine if the candidate agent added to the test well increased or decreased the activity of UBE1L. Numerous fluorescent molecules and quenchers are known in the art and are commercially available (Epoch Biosciences, Bothell, Wash., 98021; Aldrich, Milwaukee, Wis., 53201).

Methods to Alter the Level of ISG15-Conjugate

The present invention also provides a method for altering the level of ISG15-conjugated protein in a sample. In one embodiment, the method comprises contacting the sample with an effective amount of an ISG15-conjugate, or variant thereof, that alters the conjugation of ISG15 with a target protein. In another embodiment, the method comprises contacting the sample with an effective amount of an agent that alters the conjugation of ISG15 with a protein. The agent may be identified, for example, according to the methods described herein. An effective amount may be an amount that alters the level of ISG15-conjugated protein in the sample.

The present invention is also directed to a method for altering the level of ISG15-conjugated protein in a patient in need of such treatment. In one embodiment, the method comprises administering to the patient an effective amount of an ISG15-conjugate, or variant thereof, that alters the conjugation of ISG15 with a target protein. In another embodiment, the method comprises administering to the patient an effective amount of an agent that alters the conjugation of ISG15 with a target protein. The agent may be identified, for example, according to the methods described herein. An effective amount may be an amount that alters the level of ISG15-conjugated protein in the patient. The method may be used to increase or decrease the concentration of certain ISG15-conjugates in a cell of the patient.

Accordingly, the methods of the present invention may be used to treat disease characterized by an altered level of ISG15-conjugate. In a particular embodiment, the disease is interferon-related. In another particular embodiment, the disease is related to dysregulation of interferon. In another particular embodiment, the disease is responsive to interferon therapy.

Thus, in one embodiment, the present invention provides a method to determine the responsiveness of a patient to treatment with an interferon, comprising administering an interferon to the patient suspected of being responsive to interferon treatment, determining the amount of ISG-15-conjugated protein in the patient, and comparing the amount of ISG-15-conjugated protein in the patient before and after administration of the interferon, wherein an increase in ISG15-conjugated protein indicates greater responsiveness to interferon treatment.

In another particular embodiment, the disease is related to cellular proliferation. In a further embodiment, the method promotes the ISGylation of target proteins that would cause uncontrolled proliferation of the cell if they were not ISGylated by being coupled to ISG15. For example, overexpression of PLCγ1 is evident in several forms of cancer and it has been identified as a key mediator of PDGF-dependent cellular transformation. Accordingly, in a specific embodiment, the present invention provides a method to inhibit cell proliferation comprising contacting a cell with a composition comprising an ISG15-conjugate, detecting the amount of cell proliferation of the cell, and comparing the amount of cell proliferation detected in the experimental with a control amount of cell proliferation (which may be determined from the amount of proliferation of a cell not contacted with the composition), wherein a decrease in the amount of cell proliferation detected in the experimental compared to that detected in the control indicates that the composition inhibits cell proliferation.

In one non-limiting embodiment, the present invention also provides a method for treating a cell-proliferative disorder comprising administering to a patient in need thereof an effective amount of a composition comprising an agent wherein the agent increases conjugation of ISG15 with a target protein. In non-limiting embodiments, the target protein comprises Stat1, Jak1, ERK1, ERK2, and/or any combination thereof.

In another non-limiting embodiment, the present invention provides a method for treating a cell-proliferative disorder comprising administering to a patient in need thereof an effective amount of a composition comprising an ISG-conjugate. In non-limiting embodiments, the ISG15-conjugate comprises ISG15-Jak1, ISG15-ERK1, ISG15-ERK2, ISG15-Stat1, and/or any combination thereof.

In another non-limiting embodiment, the present invention provides a method for treating cancer caused by PLCγ1 overexpression by coupling the PLCγ1 to ISG15 and thereby eliminates cellular proliferation due to PLCγ1. The method may be applied to any target protein that can be coupled to ISG15 to form an ISG15-conjugate.

The agents are generally formulated with a pharmaceutically acceptable carrier and may be administered by any desired route. More particularly, the agents may be formulated with a buffered aqueous, oil or organic medium containing optional stabilizing agents and adjuvants for stimulation of immune binding. A preferred formulation involves lyophilized agent and a pharmaceutical carrier. Immediately prior to administration, the formulation is constituted by combining the lyophilized agent and pharmaceutical carrier. Administration by a parenteral or intravenous regimen will deliver the agent to the desired site of action. The dosage and route of administration will generally follow the judgment of the patient's attending physician. In some embodiments, intravenous, intraperitoneal, intramuscular, subcutaneous, rectal or vaginal administration may be used.

The amount of agent useful to establish alteration of ISG15-conjugation can be determined by diagnostic and therapeutic techniques well known to those of ordinary skill in the art. The dosage may be determined by titrating a sample of the patient's blood sera with the agent. Such titrations may be accomplished by the diagnostic techniques discussed below. The agents of the invention maybe administered in a range of about 0.001 to about 100, preferably 0.005 to about 50 mg per kg of patient body weight per day.

Pharmaceutical formulations of the agent can prepared as liquids, gels and suspensions. The formulations can be suitable for injection, insertion or inhalation. Injection may be accomplished by needle, cannula catheter and the like. Insertion may be accomplished by lavage, trochar, spiking, surgical placement and the like. Inhalation may be accomplished by aerosol, spray or mist formulation. The compound may also be administered topically such as to the epidermis, the buccal cavity and instillation into the ear, eye and nose.

The agent may be present in the pharmaceutical formulation at concentrations ranging from about 1 percent to about 50 percent, preferably about 1 percent to about 20 percent, more preferably about 2 percent to about 10 percent by weight relative to the total weight of the formulation.

The carrier for the pharmaceutical formulations includes any pharmaceutically acceptable carrier suitable for delivery by anyone of the foregoing routes and techniques of administration. Diluants, stabilizers, buffers, adjuvants, surfactants, fungicides, bactericides, and the like may also optionally be included. Such additives will be pharmaceutically acceptable and compatible with the agent. Carriers include aqueous media, buffers such as bicarbonate, phosphate and the like; ringers solution, Ficol solution, BSA solution, EDTA solution, glycerols, oils of natural origin such as almond, com, arachnis, caster or olive oil; wool fat or its derivatives, propylene glycol, ethylene glycol, ethanol, macrogols, sorbitan esters, polyoxyethylene derivatives, natural gums, and the like.

Method for Screening Patients Having a Malcondition Indicated by the Presence of a Increased or Decreased Cellular Concentration of ISG15-Conjugate

The invention provides screening methods useful for identifying patients affected with a malcondition resulting from an imbalance of the ISG15 conjugation process. Such malconditions include, for example, cell-proliferate disorders, viral diseases, and bacterial diseases. Examples of such cell-proliferative disorders include, but are not limited to, cancer.

The screening methods include those, for example, that identify antibody-antigen binding as described by the foregoing methods of the invention. An antibody of the invention can be combined with an appropriate sample from the patient to produce a complex. The complex in turn can be detected with a marker reagent that interacts with such a complex. Typical marker reagents include antibodies selective for the complex, antibodies selective for certain epitopes of the immunopolypeptide or a label attached to the immunopolypeptide itself. In particular, radioimmunoassay (RIA), radioallergosorbent test (RAST), radioimmunosorbent test (RIST), immunradiometric assay (IRMA) Farr assay, fluorescence immunoassay (FIA), sandwich assay, enzyme linked immunosorbent assay (ELISA) assay, northern or southern blot analysis, and color activation assay may be used following protocols well known for these assays. See, for example, Immunology, An Illustrated Outline by David Male, C. V. Mosby Company, St. Louis, Mo., 1986 and the Cold Spring Harbor Laboratory Manuals cited above. Labels including radioactive labels, chemical labels, fluorescent labels, luciferase and the like may also be directly attached to the immunopolypeptide.

According to the foregoing screen methods, a patient may be diagnosed as having or being at risk for developing a malcondition characterized by, or associated with, an alteration in the amount or level of ISG15-conjugation. For example, the level or amount of an ISG15-conjugate is determined in a physiological sample from a patient suspected of having or being at risk for developing a malcondition. The level or amount of conjugate from the patient can then be compared to the level or amount of an ISG15-conjugate from a subject not having, or at risk for developing, the malcondition. If the patient has an altered amount, for example, increased or decreased amount, of conjugate, then the patient may be diagnosed as having, or being at risk for developing, the malcondition. The conjugate determined may be an overall measure of ISG15-conjugate. The conjugate determined may also be a specific conjugate formed by ISG15 and a target protein, for example, phospholipase Cγ1, JAK1, ERK1, ERK2, or Stat1. The patient may then be treated by administration of compounds of the invention that alter the level or amount of ISG15-conjugate.

One specific application of the screening methods of the invention relates to screening patients for interferon hypersensitivity. Interferon is administered to patients to treat hepatitis B and hepatitis C infections. Interferon is also administered to some patients to treat hairy cell leukemia, Kaposi's sarcoma, human papillomavirus, and respiratory viruses. Accordingly, the methods of the invention may be used to determine if a particular patient may exhibit a hypersensitive response to interferon treatment because interferon hypersensitivity can be assessed through determining whether the patient exhibits increased cellular ISG15-conjugate concentration. Accordingly, a primary care physician can screen a patient through use of the methods of the invention to determine a proper dosage of interferon to administer to a patient.

Method to Increase Migration and Phagocytosis by a Cell

The present invention also provides methods to promote wound healing. The present invention also provides methods to increase migration and phagocytosis by a cell. It has been discovered that UBP43^{-/-} cells migrate faster in wound healing assays, and on transwell filters, than corresponding UBP43^{+/+} cells (see, e.g., Example 7). It has also been discovered that UBP43^{-/-} cells exhibit increased phagocytosis activity when compared to the corresponding UBP43^{+/+} cells (see, e.g., Example 8). These discoveries demonstrate a direct linkage between UBP43 activity and cell motility, phagocytosis, and wound healing. These discoveries also directly show involvement of ISGylation in regulating cell motility and phagocytosis.

Accordingly, inhibition of UBP43 activity can increase motility and phagocytosis by a cell. In addition, increased ISGylation of target proteins acting within pathways that signal cell motility and phagocytosis is thought to increase cell motility and phagocytosis. Such proteins are exemplified by ERK1, ERK2, and PLCγ1.

Accordingly, in one embodiment, the present invention provides a method to increase wound healing comprising contacting a cell with a composition comprising an UBP43 inhibitor or an ISG15-conjugate. In a particular, non-limiting embodiment, the composition further comprises a protein that participates in wound healing.

In another embodiment, the present invention provides a method to increase the motility of a cell comprising contacting the cell with a composition comprising a UBP43 inhibitor or an ISG-conjugate. In a particular, non-limiting embodiment, the composition further comprises a protein that participates in cell motility. Such methods may be used, for example, to promote wound healing and/or to stimulate immune response to a pathogen.

In another embodiment, the present invention provides a method to increase the phagocytotic activity of a cell comprising contacting the cell with a composition comprising an UBP43 inhibitor or an ISG15-conjugate. In a particular, non-limiting embodiment, the composition further comprises a protein that participates in phagocytosis. Such methods may be used, for example, to promote wound healing and to stimulate immune response to a pathogen.

In another embodiment, the present invention provides a method to modulate conjugation of ISG15 within a patient comprising administering to the patient a composition comprising an ISG-conjugate. In another embodiment, the method comprises administering to the patient a composition comprising an agent identified through use of the methods of the invention.

Therefore, administration of agents that inhibit UBP43 activity can be used to increase cell motility, cell phagocytosis, and promote wound healing. In addition, administration of ISG15-conjugates that act within a cell motility signaling pathway, such as ISG-ERK1 or ISG-ERK2, can be used to increase the motility of a cell. Administration of ISG15-conjugates that act within a phagocytosis signaling pathway, such as ISG15-PLCγ1, can be used to increase phagocytosis by a cell.

Methods to increase cell motility, cell phagocytosis, and promote wound healing have numerous applications in the treatment of injury and disease. For example, agents that increase the motility of cells may be applied to wounds to increase physical repair by stimulating migration of cells, such as fibroblasts. These agents may also stimulate innate and specific immune responses within a wound by stimulating migration of cells of the immune system to the wound. Innate immunity includes phagocytic cells, such as neutrophils and macrophages, and other leukocytes, such as natural killer cells. Specific immunity responds to specific antigens and includes such cells as lymphocytes and their products. These cells may also stimulate wound healing by reducing or eliminating infections due to exposure to pathogens occurring during a wounding event, such as bacteria and viruses. The phagocytic activity used by such cells to engulf such pathogens may be increased by administering agents that inhibit, UBP43 activity or by administering ISG15-conjugates, such as ISG15-PLCγ1.

Agents which inhibit UBP43 activity, or ISG15-conjugates, may be prepared in many different types of formulations that can be administered to a patient in need thereof. For example, agents and conjugates may be prepared as salves, creams, lotions, and the like for topical administration to a specific area. Agents and conjugates may be included in bandages, sutures, implants, artificial skin, or other medical devices that are placed on or within a patient. Examples of such implants include artificial joints, artificial bone, natural bone, and the like. It is also envisioned that such agents and conjugates may be prepared as extended release formulations. Such formulations include bioerodible implants that are formed upon injection of a suitable polymer into a patient. Many of these types of implants are known and have been described (U.S. Pat. Nos.: RE37,950; 6,461,631; 6,261,583; 6,143,314). Methods to prepare pharmaceutical formulations and to determine dosages are known in the art and are disclosed herein.

### EXAMPLES

### Example 1

### Preparation of Antibodies to ISG15

Hybridoma (clone 2.1) (24) that produces monoclonal mouse anti-human ISG15 antibodies was cultured in Hybridoma-SFM medium (Invitrogen, Carlsbad, Calif.). Cells were removed by centrifugation at 2,000×g for 5 minutes, and the supernatants were filtered through a 0.22 μm filter to remove debris. IgGs were purified on a protein G column (Amersham Biosciences Inc., Piscataway, N.J.) according to the manufacturer's instructions. The eluted IgGS were then dialyzed against coupling buffer (0.2 M Na₂HPO₄, 0.2 M NaCl, pH 8.5). Six mg of purified IgGs were mixed with 1 ml of glyoxal activated agarose (Active Motif, Carlsbad, Calif.) and NaBH₃CN (Sigma, St. Louis, Mo.) to a final concentration of 50 mM. Coupling proceeded overnight at 24° C. with constant rocking. Coupled resin was removed and the supernatant was allowed to couple with a fresh batch of glyoxal activated agarose. Success of immobilization was determined by measuring protein concentration in the buffer before and after coupling and was estimated to be 2.6 and 1.6 mg IgG per ml of resin after first and second coupling respectively. Unreacted sites on coupled resin were blocked with 10 mm Methanolamine, pH 8.0 (2 h at 24° C.). IgG-resin was washed sequentially with 10 volumes of each of the following: PBS, 1% Triton X-100 in PBS, PBS, 2 M NaCl in PBS, PBS and stored in PBS containing 0.01% Thimerosal (Sigma) at 4° C. Immediately before use in immunoaffinity purification of ISG15-conjugates, the IgG-resin was washed with 10 volumes of 0.1 M glycine, pH 2.5.

### Example 2

### Obtaining Samples to be Tested for ISG15-Conjugates

According to appropriate sample gathering protocols, thymus samples were obtained (with ethical approval) from children (aged 1 to 10 years) during routine cardiac surgery. Nine grams of tissue was macerated with a razor blade and homogenized using a tissue homogenizer in 25 ml of RIPA buffer (50 mM Tris, pH 7.5, 5 mM EDTA, 150 mM NaCl, 0.1% w/v SDS, 1% v/v Triton X-100, 0.5% w/v sodium deoxycholate, 2 mM phenylmethylsulfonyl fluoride, and protease inhibitor mixture (Sigma, product number P2714)). The slurry was sonicated (four 30-s pulses), and insoluble material was removed by 10 minutes centrifugation at 18,000×g. This extraction procedure yielded 500 mg of total protein. Supernatant was stored at −80° C. and passed through a 0.4 μm filter immediately before use in immuno-affinity purification.

A series of pilot experiments established that certain ISG15-conjugates would bind to IgG-resin inefficiently at pH 8.0, but bound efficiently at pH 7.0. The addition of ethylene glycol improved conditions for binding of an overlapping but distinct set of ISG15-conjugates. Combination of pH 7.0 with ethylene glycol, on the other hand, resulted in massive binding of certain nonspecific proteins. Therefore, the following two-step protocol was adopted. IgG-resin (0.5 ml) was mixed with thymic protein extract (250 mg total protein, at a final concentration of 3 mg/ml) in RIPA buffer (pH 7.0) to form a final volume of 83 ml. Binding of ISG15-conjugates was carried out overnight at 4° C. The resin was then washed 3 times with 20 volumes of RIPA buffer pH 7.0 and stored at −20° C. The protein extract was adjusted to pH 8.0 and ethylene glycol was added to a final concentration of 25% v/v. Fresh IgG-resin was added and binding was carried out as above. The resin was washed 3 times with 20 volumes of RIPA buffer pH-8.0, 25% ethylene glycol and stored at −20° C. Bound proteins resulting from both steps were eluted from IgG-resins by the addition of 2 volumes of SDS-PAGE loading buffer and a 5-min incubation at 90° C. The ISG15-conjugates from both binding steps (pH 8.0 and pH 7.0, 25% ethylene glycol) were pooled and analyzed by high-throughput western blot.

### Example 3

### Identification of ISG15 Target Proteins

A protein modified by ISG15 acquires two new characteristics: it gains affinity to anti-ISG15 antibodies and it migrates slower on SDS-PAGE. Using these features, ISG15 target proteins were identified. High throughput western blot (PowerBlot) was originally developed for comparative analysis of two or more samples (e.g., protein extracts from normal and diseased tissues) with the intent to identify proteins whose levels are altered. This technique was a useful tool for identifying post-translationally modified proteins in samples enriched for the modified species. Additionally, several improvements to the high-throughput western blot technology are disclosed herein. First, although post-surgical thymus was a good source of ISG15-conjugated proteins, the amount of ISG15-conjugates in this tissue was approximately three times lower than in IFN-treated A549 or U937 cells. Second, a significant amount of free ISG15 that was present in cells and tissues strongly bound to immobilized anti-ISG15 antibodies, occupied valuable sites and decreased efficiency of purification of ISG15-conjugates. Free ISG15, however, was efficiently removed by gel filtration chromatography on Sephadex G-50. Third, false-positives detected in the primary screening step, which resulted from nonspecific binding to IgG-resin, were eliminated if simultaneous immuno-affinity purification was performed on immobilized anti-ISG15 and isotype control IgG. Proteins absorbed to both resins were then analyzed on parallel blots, and nonspecific bands were identified. Fourth, the sensitivity of the method was increased more than 10-fold since only 0.5 μg of total protein was loaded per lane according to the method described herein, while PowerBlot high-throughput western blotting permits loading of up to 7.5 μg of total protein per lane.

ISG15-conjugated proteins were purified from human thymus by immuno-affinity chromatography, and the ISG15-conjugates were analyzed by high-throughput western blot. ISG15-conjugated proteins purified from human thymus by immunoaffinity chromatography were analyzed by SDS-PAGE and high-throughput western blot. The assay used 860 individual monoclonal antibodies (mAbs), of which 710 cross-react with human proteins. Some of the mAbs were different clones recognizing the same protein or phosphorylated versions of the same protein. 645 individual proteins can be detected by this technology.

Primary screening of the purified ISG15-conjugates was performed at BD Biosciences Transduction Laboratories (Lexington, Ky.). Briefly, the purified ISG15-conjugates were separated on six high-resolution gradient gels and transferred onto nitrocellulose membranes. Each membrane was divided into 40 lanes by applying a chamber-forming grid. To each chamber a cocktail of mAbs was added (1-8 mAbs per cocktail; idenfities of mAbs and location with respect to lanes and templates are available from BD Biosciences Transduction Laboratories). After incubation for one hour at room temperature, the chambers were rinsed and incubated with secondary antibodies under the same conditions (Alexa 680-labeled goat anti-mouse IgGs; Molecular Probes, Eugene, Oreg.). Images were then acquired with an infra-red scanner (Li-Cor, Lincoln, Nebr.). The bands were identified and molecular masses assessed using specialized software by PowerBlot service. Each observed band, where possible, was manually matched against the expected molecular mass of a protein recognized by an individual antibody in the cocktail. The computer-generated data were scrutinized in our laboratory by careful manual analysis and were found to be more than 90% accurate.

Computer-assisted and subsequent manual examination of detected signals revealed 149 bands. The bands were separated into three groups based on the confidence with which the identity of the protein could be deduced. Group one (72 bands) included proteins that (a) matched very well the expected molecular mass of unmodified proteins and (b) were unlikely to be the result of a smaller protein which has been modified by ISG15. Proteins of this group were thought to be nonspecifically bound to IgG-resin (either agarose itself or IgGs). However, the proteins may also have been co-purified with ISG15-conjugated protein(s). Group two (73 bands) incorporated the bands of ambiguous identity. The apparent molecular masses of group two bands corresponded to an expected molecular mass of an unmodified protein, but may correspond to a molecular mass of another protein modified by one or two molecules of ISG15.

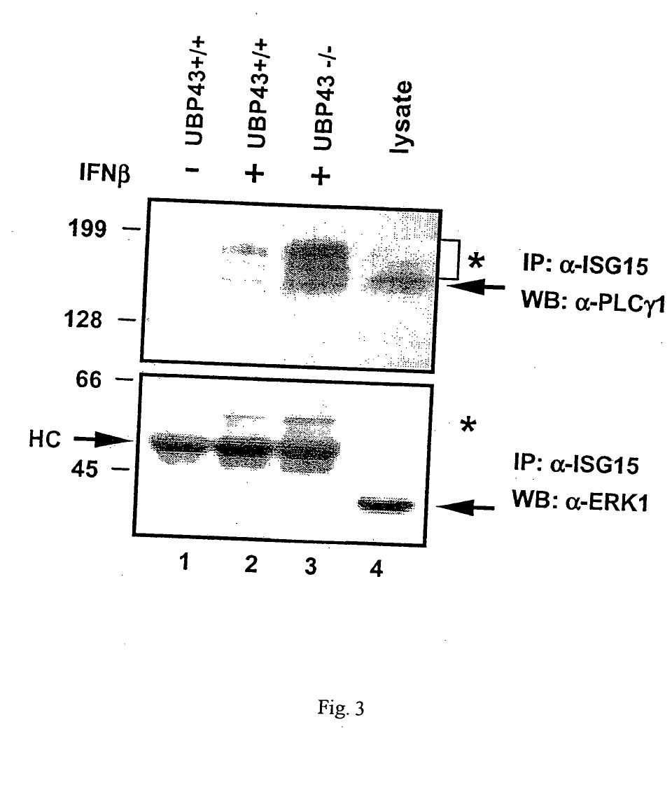

Molecular masses of group 3 proteins (three bands or groups of bands) did not correspond to any protein and were considered as the candidates likely to be ISG15-modified. FIG. 1 shows three fragments of respective western blot panels. PLCγ1 was represented by four bands migrating in the range of 145-185 kDa (fragment A), whereas ERK1 and JAK1 were represented by single bands of 57 and 145 kDa, respectively. The molecular masses of these bands (with the exception of 145 kDa band recognized by PLCγ1, which may be a product of proteolysis; see FIGS. 2 and 3) were in good correlation with expected shifts in mobility upon addition of one or more molecules of ISG15.

### Example 4

### Immunoprecipitations and Western Blots

To verify the results of the primary screen, immunoprecipitations (IPs) were performed with additional controls as well as reciprocal IPs. Anti-Stat1 (rabbit polyclonal, anti-peptide) antibodies were purchased from Santa Cruz Biotechnology (Santa Cruz, Calif.). Anti-ERK1, anti-PLCγ1 and anti-JAK1 mAbs were purchased from BD Biosciences Transduction Laboratories. Anti-ERK1 mAbs (catalog no. 610030) recognize both 44 and 42 kDa bands that may represent ERK1 and ERK2, respectively. Therefore, the ISG15-modified protein identified may also be ERK2. Rabbit anti-human ISG15 polyclonal antibodies were previously described (24). Rabbit anti-mouse ISG15 polyclonal antibodies were generated as follows. Murine ISG17 (pro-ISG15) was PCR-amplified (primers TGGAATTCTTAGGCACACTGGTCCCC (SEQ ID NO: 1) and AATTCGATTCTGGATCCGCCTGGGACC (SEQ ID NO.2)) from a cDNA library prepared from HBV infected hepatocytes and cloned into pGEM-T Easy vector (Promega, Madison, Wis.). The correct sequence was verified by sequencing, the insert excised with BamH I and Sal I restriction endonucleases, and recloned into respective sites of pQE-30 vector (Qiagen, Valencia, Calif.) to yield 6His-tagged ISG17. 6His-ISG17 was then expressed in E. coli and purified on Ni-NTA resin (Qiagen) as recommended by the manufacturer. Rabbit sera were generated by Covance (Denver, Pa.). Anti-ISG15 specific IgGS were immunoaffinity purified on immobilized 6His-ISG15 and depleted against immobilized Ub (Sigma) to remove any cross reacting IgGs. 6His-ISG15 and Ub were immobilized on glyoxal activated agarose as described above for mAbs.

With the exception of anti-ERK1 (lysate was prepared in denaturing conditions as recommended by the manufacturer of mAbs) all lysates were prepared and IPs were performed in RIPA buffer. Protein A Sepharose was used with polyclonal antibodies and Protein G Sepharose was used with mAbs. Western blotting was performed as previously described (12).

In order to validate the results of high-throughput western blot analysis, immunoprecipitations (IPs) were performed with anti-ISG15 mAb. The immunocomplexes were analyzed by western blot with anti-PLCγ1, anti-JAK1 and anti-ERK1 mAbs. Bands identical to those observed on PowerBlot images were detected only when specific anti-ISG15 mAb, but not when an isotype control IgG, were used for IP (FIG. 2A). Reciprocal IPs with individual mAbs against PLCγ1, JAK1 and ERK1 were performed (FIG. 2B). Immunocomplexes were analyzed by western blot with polyclonal rabbit anti-human ISG15 antibodies and, again, the signals corresponding to ISGylated proteins were detected. These results show that PLCγ1, JAK1 and ERK1 are ISG15-conjugated proteins, and thus serve as ISG15 target proteins.

### Example 5

### Analysis of ISG15-Conjugates in UBP43 Knockout Mice

Wild type and UBP43^{-/-} murine embryonic fibroblasts (MEFs) were generated from a litter of embryos on embryonic day 12.5. Briefly, embryonic torsos were minced and trypsinized for 30 min at 37° C. Cells were harvested, resuspended in DMEM supplemented with 10% fetal bovine serum (FBS), and plated on 10 cm dishes. MEFs (9×10⁵) were plated on 60 mm plates and replated every three days for over 20 passages until they immortalized. Where indicated MEFs were treated with 200 units/ml of IFNβ, 5 μM lactacystin, and 10 μM MG132 (Calbiochem, La Jolla, Calif.). Thymocytes and bone marrow cells were isolated from 4-6 week old mice and were cultured in RPMI 1640 supplemented with 10% FBS and 100 U/ml of Penicillin and 100 μg/ml of Streptomycin (Life Technologies, Rockville, Md.). Bone marrow cultures were also supplemented with the following growth factors: 10 ng/ml IL-3, 10 ng/ml IL-6, and 100 ng/ml stem cell factor (SCF) (PeproTech, Rocky Hill, N.J.).

A knockout mouse model was developed in which a gene coding for the ISG15-specific protease UBP43 is deleted (19). Most tissues of UBP43^{-/-} mice had a large increase of ISG15-conjugates, relative to wild type. This difference can be further increased following dosing with LPS or poly(I—C). MEFs derived from UBP43^{-/-} and UBP43^{+/+} mice were used to assess the relevance of our findings in human tissue, to the murine model. MEFs of both genotypes were incubated with IFNβ for 24 hr. ISG15-conjugated proteins were immunoprecipitated with purified, rabbit anti-mouse ISG15 antibodies and immunocomplexes were analyzed by western blot. The results presented in FIG. 3 demonstrate that murine PLCγ1 and ERK1 are also modified by ISG15. The modification, however, was only obvious after IFNβ treatment and, as expected, was stronger in the UBP43^{-/-} cells. The band of 145 kDa, that was detectable with anti-PLCγ1 in immunoprecipitates from human thymus, was also present in MEFs. This band is thought to be a product of specific proteolysis of ISG15-conjugated PLCγ1. Detection of ISGylation of JAK1 in MEFs was not demonstrated, however, the modification was detectable in murine thymocytes suggesting that the set of ISG15-conjugated proteins may be cell-15 specific. Stat1, a transcription factor and substrate of JAK1, remains activated for a longer period of time in UBP43^{-/-} cells (20). In many western blot experiments, where UBP43^{-/-} animals or isolated cells were challenged with Jak-Stat activators, the appearance of higher molecular weight bands recognized by Stat1 antibodies was observed (FIG. 4A). These high molecular weight bands may have been Stat1 molecules conjugated by ISG15. Reciprocal immunoprecipitations and western blotting with rabbit polyclonal antibodies against Stat1 and ISG15 revealed the presence of up to five specific bands which are thought to be Stat1-ISG15 conjugates (FIG. 4B). The two bands (Stat1α/β, 91 and 84 kDa), corresponding to unmodified Stat1, are non-specifically recognized by ISG15 antibodies due to the overload of Stat1 from immunoprecipitation using Stat1 antibodies. These two bands also appeared on the membrane with Ponceau-S staining (FIG. 4B, left panels). The same bands corresponding to unmodified Stat1α/β are detectable with anti-Stat1 in the proteins immunoprecipitated with anti-ISG15 (FIG. 4B, right panels). Stat1 can form homodimers (25), and the copurification of unmodified molecules may be caused by protein-protein interaction between ISG15-conjugated and unconjugated Stat1. In addition, other proteins that interact with Stat1 to form ISGF3 and other complexes may be ISG15-conjugated and cause the copurification of unmodified Stat1.

Western blotting with anti-Stat1 on purified ISG15-conjugates was performed to determine whether Stat1 is ISG15-conjugated in human thymus. The same preparation of antibodies that was used for PowerBlot analysis was used (FIG. 4C). A banding pattern similar to that observed in murine T-cells (FIG. 4B) was detected.

### Example 6

### Proteasome Inhibitors do not Increase the Amount of ISG15-Conjugates

The major function of ubiquitination is localization of targeted proteins to proteasomes for subsequent degradation (26; 27). It was not known whether conjugated ISG15 acted like ubiquitin to direct conjugated proteins for proteasomal degradation. Accordingly, due to their absence of ISG15-deconjugation, UBP43^{-/-} cells were used to analyze the relationship between Ub, ISG15, and the proteosome.

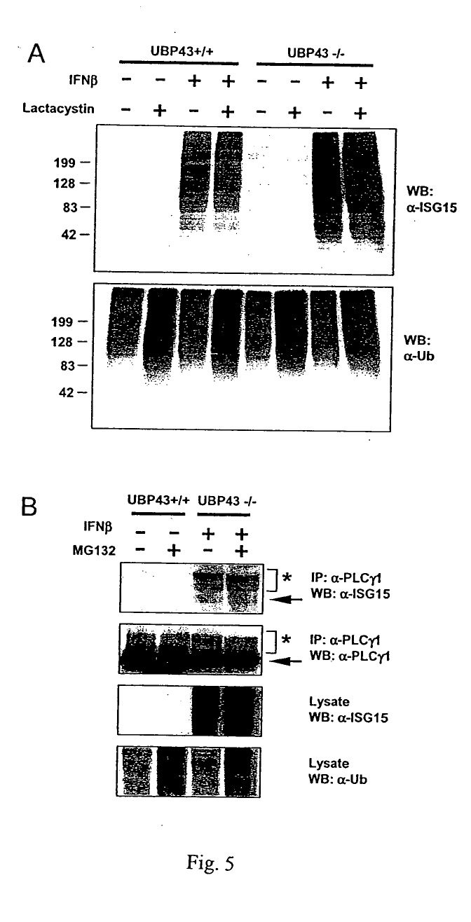

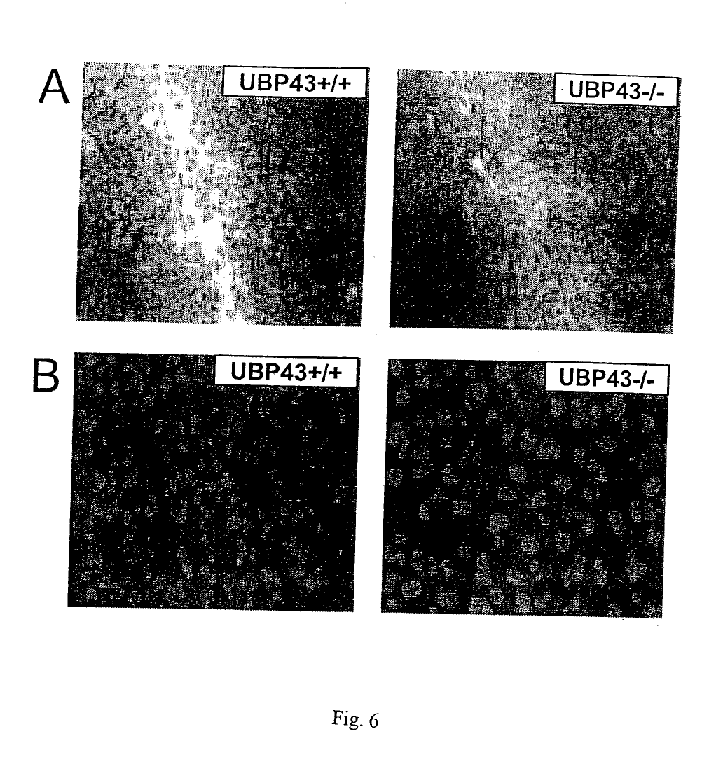

UBP43^{-/-} and UBP43^{+/+} cells, uninduced or IFNβ induced, were treated with a highly specific proteasome inhibitor lactacystin. Western blot revealed no difference in the amount of ISG15-conjugates between lactacystin-treated and untreated samples, while, as expected, an increase in the amount of ubiquitinated proteins was observed (FIG. 5A). Noticeably, the level of ISG15 conjugation had no detectable effect on the appearance of Ub-conjugates in either UBP43^{-/-} and UBP43^{+/+} cells treated with IFNβ. To confirm this observation, PLCγ1 was immunoprecipitated from MEFs treated with a proteasome inhibitor and immunocomplexes were probed with anti-ISG15 antibodies (FIG. 5B). Consistent with the data of total protein analysis, no increase in the amount of ISGylated PLCγ1 was observed upon inhibition of proteasomes. These results demonstrate that ISG15-conjugated proteins are not degraded by proteasomes and indicate that ISGylation does not prevent conjugation of Ub.

### Example 7

### UBP43 and ISG15 Modification in Interferon JAK-STAT Signaling

ISG15 is a ubiquitin-like protein containing two ubiquitin homology domains in tandem. ISG15 conjugates to a variety of proteins in cells treated with type I interferon or lipopolysaccharide. Although ISG15 was identified more than two decades ago and is one of the highly expressed proteins in most cell types challenged by virus or bacteria, the biochemical and physiological functions of ISG15-conjugation to proteins are unknown. Analysis of UBP43, a protease that specially removes ISG15 from ISG15-conjugated proteins, has shown that dysregulation of protein ISG15-conjugation in mice leads to decreased life expectancy and brain cell injury. This indicates that balanced levels of protein ISG15 modification are important for normal cellular function. Furthermore, UBP43 knockout mice are hypersensitive to type I interferon stimulation. In UBP43 deficient cells, interferon induces a prolonged Stat1 tyrosine phosphorylation, DNA binding, and interferon mediated gene activation, indicating the prolonged and enhanced interferon signaling relative to control cells.

The expression and the modification of important players in type I interferon signaling, including JAK1, Stat1, SOCS proteins, CD45, PTP-1B, TC-PTP, SHP-1 and SHP-2 was analyzed to investigate the molecular basis of such altered activation of Jak-Stat signaling in UBP43 deficient cells. Two major components of Jak-Stat signaling pathway, JAK1 and Stat1, are modified by ISG15. Additionally, an ISG15-activating enzyme, UBE1L defective human leukemia cell line (K562) has been identified. K562 cells do not show protein ISG15 conjugation upon interferon treatment despite the significant induction of free ISG15 expression. Restoration of ISG15 conjugation in K562 cells significantly increases interferon stimulated promoter activity. Taken together, these findings identify UBP43 as a novel negative regulator of interferon signaling and suggest the involvement of protein ISGylation in the regulation of the JAK-STAT pathway.

### Example 8

### UBP43−/− Cells Exhibit Increased Migratory Activity

UBP43 knockout mice demonstrated increased activity of the IFN signaling Jak-Stat pathway in bone-marrow cells. Both JAK1 (a kinase) and Stat1 (a substrate of JAK1 and a component of the ISGF3 transcription complex) were modified by ISG15. Such ISGylation of JAK1 and Stat1 is thought to be directly involved in IFN induced signaling.

The ability of UBP43^{-/-} mouse embryonic fibroblasts (MEFs) to migrate was tested through use of a wound healing assay. Primary MEFs that were used in migration assays were of passage one to six. Peritoneal macrophages used in migration assays were prepared as previously described (Malakhova et al., J. Biol. Chem., 277: 14703 (2002)). The peritoneal macrophages were plated on 8-well glass chamber slides (Nalge Nunc, Rochester, N.Y.). Wound healing assay protocols are known and have been described (Xu et al., Development, 125: 327 (1998)). The wound healing assay with primary MEFs demonstrated that UBP43^{-/-} cells migrated faster than UBP43^{+/+} cells (FIG. 5A). Migration assays were also conducted on transwell filters (Corning Costar Corp., Cambridge, Mass.).

These assays demonstrated that UBP43^{-/-} primary MEFs and peritoneal macrophages extracted from UBP43^{-/-} mice displayed increased migratory activity relative to the corresponding UBP43⁺⁺ cells.

### Example 9

### UBP43−/− Cells Exhibit Increased Phagocytic Activity

The phagocytic activity of UBP43^{-/-} peritoneal macrophages was tested with zymosan particles from Molecular Probes that were covalently labeled with Texas Red (FIG. 5B). These peritoneal macrophages were prepared as described above. The zymogen particles were prepared and opsonized with normal mouse IgG. Macrophages were plated on glass chambers and allowed to adhere for 1 hour. The cells were allowed to ingest the opsonized zymosan particles for 15 minutes at 37° C. Uningested particles were quenched with Trypan Blue (Sigma, St. Louis, Mo.). Opsonization and quenching were performed according to manufacturers instructions (Molecular Probes, Eugene, Oreg.).

UBP43^{-/-} macrophages were 48% more active and ingested 6.7 particles per cell while UBP43^{+/+} macrophages injested 4.5 particles (assumed 100%). Controls performed at 4° C. in which phagocytosis was suppressed but non-specific binding of zymosan particles to the cell surface still occurred revealed no difference. These results confirmed that increased phagocytic activity increased in UBP43^{-/-} macrophages.

Documents
1. Farrell, P. J., Broeze, R. J., and Lengyel, P. (1979) Nature 279,523-525
2. Der, S. D., Zhou, A., Williams, B. R., and Silverman, R. H. (1998) Proc.Natl.Acad.Sci. U.S.A. 95, 15623-15628
3. Haas, A. L., Ahrens, P., Bright, P. M., and Ankel, H. (1987) J. Biol Chem. 262, 11315-11323
4. Li, J., Peet, G. W., Balzarano, D., Li, X., Massa, P., Barton, R. W., and Marcu, K. B. (2001) J. Biol. Chem. 276, 18579-18590
5. D'Cunha, J., Knight, E. Jr., Haas, A. L., Truitt, R. L., and Borden, E. C. (1996) Proc.Natl.Acad.Sci. U.S.A. 93,211-215
6. Hansen, T. R., Austin, K. J., Perry, D. J., Pru, J. K., Teixeira, M. G., and Johnson, G. A. (1999) J Reprod.Fertil.Supp 154, 329-339
7. Loeb, K. R. and Haas, A. L. (1992) J. Biol. Chem. 267, 7806-7813
8. Pickart, C. M. (2001) Annu.Rev.Biochem. 70, 503-533
9. Ciechanover, A., Orian, A., and Schwartz, A. L. (2000) Bioessays 22, 442-451
10. Narasimhan, J., Potter, J. L., and Haas, A. L. (1996) J. Biol. Chem. 271,324-
11. Yuan, W. and Krug, R. M. (2001) EMBO J. 20,362-371
12. Malakhov, M. P., Malakhova, O. A., Kim, K. I., Ritchie, K. J., and Zhang, D. E. (2002) J. Biol. Chem. 277, 9976-9981
13. Liu, L. Q., Ilaria, R., Jr., Kingsley, P. D., Iwama, A., van Etten, R. A., Palis, J., and Zhang, D. E. (1999) Mol. Cell Biol 19, 3029-3038
14. Schwer, H., Liu, L. Q., Zhou, L., Little, M. T., Pan, Z., Hetherington, C. 1., and Zhang, D. E. (2000) Genomics 65, 44-52
15. Zhang, X., Shin, J., Molitor, T. W., Schook, L. B., and Rutherford, M. S. (1999) Virology 262, 152-162
16. Li, X. L., Blackford, J. A., Judge, C. S., Liu, M., Xiao, W., Kalvakolanu, D. V., and Hassel, B. A. (2000) J. Biol. Chem. 275, 8880-8888
17. Kang, D., Jiang, H., Wu, Q., Pestka, S., and Fisher, P. B. (2001) Gene 267, 233-242
18. Malakhova, O., Malakhov, M., Hetherington, C., and Zhang, D. E. (2002) J. Biol. Chem. 277, 14703-14711
19. Ritchie, K. J., Malakhov, M. P., Hetherington, C., Zhou, L., Little, M. T., Malakhova, O. A., Sipe, J., and Zhang, D. E. (2002) Genes and Development 16, 2207-2212
20. Malakhova, O., Yan, M., Malakhov, M. P., Yuan, Y., Ritchie, K. J., Kim, K. I., Peterson, L. F., Shuai, K., and Zhang, D. E. (2002) Genes and Development in press,
21. Hochstrasser, M. (2000) Science 289,563-564
22. Jentsch, S. and Pyrowolakis, G. (2000) Trends Cell Biol 10, 335-342
23. Hamerman, J. A., Hayashi, F., Schroeder, L. A., Gygi, S. P., Haas, A. L., Hampson, L., Coughlin, P., Aebersold, R., and Aderem, A. (2002) J. Immunol. 168, 2415-2423
24. D'Cunha, J., Ramanujam, S., Wagner, R. J., Witt, P. L., Knight, E. Jr., and Borden, E. C. (1996) J. Immunol. 157, 4100-4108
25. Leonard, W. J. and O'Shea, J. J. (1998) Annu.Rev. Immunol.16, 293-322.
26. Coux, O., Tanaka, K., and Goldberg, A. L. (1996) Annu.Rev.Biochem. 65, 801-847
27. Varshavsky, A. (1997) Trends Biochem.Sci. 22, 383-387
28. Knight, E. Jr. and Cordova, B. (1991) J. Immunol. 146, 2280-2284.
29. Rhee, S. G. (2001) Annu.Rev.Biochem. 70, 281-312
30. Rebecchi, M. J. and Pentyala, S. N. (2000) Physiol Rev. 80, 1291-1335 31.
31. Carpenter, G. and Ji, Q. (1999) Exp. Cell Res. 253, 15-24
32. Ji, Q. S., Ennini, S., Baulida, J., Sun, F. L., and Carpenter, G. (1998) Mol. Biol. Cell 9, 749-757
33. DeMali, K. A., Whiteford, C. C., Ulug, E. T., and Kazlauskas, A. (1997) J. Biol. Chem. 272,9011-9018
34. Muller, M., Briscoe, J., Laxton, C., Guschin, D., Ziemiecki, A., Silvennoinen, O., Harpur, A. G., Barbieri, G., Witthuhn, B. A., Schindler, C., and (1993) Nature 366, 129-135
35. Rodig, S. J., Meraz, M. A., White, J. M., Lampe, P. A., Riley, J. K., Arthur, C. D., King, K. L., Sheehan, K. C., Yin, L., Pennica, D., Johnson, E. M., Jr., and Schreiber, R. D. (1998) Cell 93, 373-383
36. Durbin, J. E., Hackenmiller, R., Simon, M. C., and Levy, D. E. (1996) Cell 84, 443-450
37. Meraz, M. A., White, J. M., Sheehan, K. C., Bach, E. A., Rodig, S. J., Dighe, A. S., Kaplan, D. H., Riley, J. K., Greenlund, A. C., Campbell, D., CarverMoore, K., DuBois, R. N., Clark, R., Aguet, M., and Schreiber, R. D. (1996) Cell 84, 431-442
38. Howe, A. K., Aplin, A. E., and Juliano, R. L. (2002) Curr.Opin.Genet.Dev. 12, 30-35
39. English, J. M. and Cobb, M. H. (2002) Trends Pharmacol.Sci. 23,40-45
40. McLaughlin, P. M., Helfrich, W., Kok, K., Mulder, M., Hu, S. W., Brinker, M. G., Ruiters; M; H., de Leij, L F., and Buys, C. H. (2000) Int. J. Cancer 85, 871 876

All publications, patents and patent applications are incorporated herein by reference in their entirety. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to identify an ISG15 target protein, comprising:
(a) contacting a sample with an antibody, wherein the sample is suspected of having a conjugate comprising ISG15 and a target protein under conditions that permit the antibody to bind the conjugate; and
(b) detecting the target protein of the bound conjugate, wherein the target protein is an ISG15 target protein.

2. The method of claim 1, wherein step (a) comprises:
(a) contacting the sample with a first antibody that binds to the conjugate to form a first complex; and
(b) contacting the conjugate with a second antibody that binds to the conjugate to form a second complex.

3. The method of claim 2, wherein the first antibody is separated from the conjugate prior to contacting the conjugate with the second antibody.

4. The method of claim 2, wherein the first antibody binds to ISG15 when bound to the target protein and the second antibody binds to the target protein when bound to ISG15.

5. The method of claim 2, wherein the first antibody binds to the target protein when bound to ISG15 and the second antibody binds to ISG15 when bound to the target protein.

6. The method of claim 1, wherein the antibody selectively binds to the conjugate.

7. The method of claim 1, wherein the sample is a physiological sample obtained from a mammal.

8. The method of claim 7, wherein the mammal is a human.

9. The method of claim 7, wherein the sample is from thymus.

10. The method of claim 1, further comprising separating unconjugated ISG15 from the sample prior to contacting the sample with the antibody.

11. The method of claim 10, wherein the unconjugated ISG15 is removed from the sample by gel filtration chromatography.

12. The method of claim 11, wherein the unconjugated ISG15 is removed from the sample by gel filtration chromatography on Sephadex G-50.

13. The method of claim 1, wherein the sample is prepared in vitro by combining ISG15, an ISG15 activator enzyme, and an ISG15 target protein to form a conjugate comprising ISG15 and the target protein.

14. The method of claim 1, wherein the ISG15 target protein is detected by western blot.

15. The method of claim 6, wherein the antibody is a monoclonal antibody.

16. The method of claim 2, wherein the first antibody is selected from the group consisting of a monoclonal antibody and a polyclonal antibody.

17. The method of claim 2, wherein the second antibody comprises a monoclonal antibody and a polyclonal antibody.

18. The method of claim 2, wherein the second antibody comprises a mixture of antibodies that bind to the target protein when bound to ISG15.

19. The method of claim 2, wherein the first antibody is immobilized.

20. A method to identify an ISG15 target protein, comprising:
(a) isolating a conjugate comprising ISG15 and a target protein; and
(b) identifying the target protein.

21. The method of claim 20, wherein the conjugate of ISG15 and a target protein is isolated through use of an antibody, or liquid chromatography.

22. The method of claim 20, wherein mass spectrometry is used to detect the target protein.

23. The method of claim 20, further comprising separating ISG15 from the target protein.

24. The method of claim 23, wherein UBP43 is used to separate ISG15 from the target protein.

25. A method to identify a compound that alters the conjugation of ISG15 with a target protein, comprising:
(a) contacting a sample comprising ISG15, an ISG15 activating enzyme, and an ISG15 target protein with a compound;
(b) detecting the amount of the conjugate in the sample; and
(c) comparing the amount of conjugate detected in step (b) with a control amount of conjugate, as determined from a sample not contacted with the compound, wherein a difference in the amount of conjugate detected in step (b) and the control amount indicates that the compound alters the conjugation of ISG15 with the target protein.

26. The method of claim 25, wherein the detection step further comprises:
(a) contacting the sample with a first antibody that binds to the conjugate to form a first complex; and
(b) contacting the conjugate with a second antibody that binds to the conjugate of ISG15 and the target protein to form a second complex.

27. The method of claim 26, wherein the first antibody is separated from the conjugate prior to contacting the conjugate with the second antibody.

28. The method of claim 26, wherein the first antibody binds to ISG15 when bound to the target protein and the second antibody binds to the target protein when bound to ISG15.

29. The method of claim 26, wherein the first antibody binds to the target protein when bound to ISG15 and the second antibody binds to the ISG15 when bound to the target protein.

30. The method of claim 25, wherein the amount of conjugate is detected by contacting the sample with an antibody that selectively binds to the conjugate.

31. The method of claim 25, wherein the sample is a physiological sample obtained from a mammal.

32. The method of claim 31, wherein the mammal is a human.

33. The method of claim 31, wherein the sample is from thymus.

34. The method of claim 25, further comprising separating unconjugated ISG15 from the sample prior to detecting the amount of conjugate in the sample.

35. The method of claim 34, wherein the unconjugated ISG15 is removed from the sample by gel filtration chromatography.

36. The method of claim 35, wherein the unconjugated ISG15 is removed from the sample by gel filtration chromatography on Sephadex G-50.

37. The method of claim 25, wherein the sample is prepared in vitro.

38. The method of claim 25, wherein the ISG15 target protein is detected by western blot.

39. The method of claim 30, wherein the antibody is selected from the group consisting of a monoclonal antibody and a polyclonal antibody.

40. The method of claim 26, wherein the first antibody is selected from the group consisting of a monoclonal antibody and a polyclonal antibody.

41. The method of claim 26, wherein the second antibody is selected from the group consisting of a monoclonal antibody or a polyclonal antibody.

42. The method of claim 26, wherein the second antibody comprises a mixture of antibodies that bind to the target protein when bound to ISG15.

43. The method of claim 26, wherein the first antibody is immunobilized.

44. The method of claim 25, wherein the sample further comprises UBP43.

45. A composition comprising an isolated ISG15-conjugate.

46. The composition of claim 45, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

47. The composition of claim 45, further comprising a pharmaceutically acceptable carrier.

48. A method to diagnose a patient having a malcondition characterized by an altered level of ISG15-conjugated protein, comprising:
(a) contacting an antibody that selectively binds an ISG15-conjugated protein with a sample from the patient suspected of having an ISG15-conjugated protein; and
(b) detecting the level of ISG15-conjugated protein in the sample, wherein an alteration in the level of ISG15-conjugated protein, as compared to the level of ISG15-conjugated protein in a patient not having the malcondition, indicates that the patient has the malcondition.

49. The method of claim 48, wherein step (a) comprises:
(a) contacting the sample with a first antibody that binds to the conjugate to form a first complex; and
(b) contacting the conjugate with a second antibody that binds to the conjugate to form a second complex.

50. The method of claim 49, wherein the first antibody is separated from the conjugate prior to contacting the conjugate with the second antibody.

51. The method of claim 49, wherein the first antibody binds to ISG15 when bound to the target protein and the second antibody binds to the target protein when bound to ISG15.

52. The method of claim 49, wherein the first antibody binds to the target protein when bound to ISG15 and the second antibody binds to the ISG15 when bound to the target protein.

53. The method of claim 48, wherein the antibody selectively binds to the conjugate.

54. The method of claim 48, wherein the sample is from thymus.

55. The method of claim 48, further comprising separating unconjugated ISG15 from the sample prior to contacting the sample with the antibody.

56. The method of claim 55, wherein the unconjugated ISG15 is removed from the sample by gel filtration chromatography.

57. The method of claim 56, wherein the unconjugated ISG15 is removed from the sample by gel filtration chromatography on Sephadex G-50.

58. The method of claim 48, wherein the ISG15 target protein is detected by western blot.

59. The method of claim 58, wherein the antibody comprises a monoclonal antibody.

60. The method of claim 49, wherein the first antibody is selected from the group consisting of a monoclonal antibody and a polyclonal antibody.

61. The method of claim 49, wherein the second antibody is selected from the group consisting of a monoclonal antibody and a polyclonal antibody.

62. The method of claim 49, wherein the second antibody comprises a mixture of antibodies that bind to the target protein when bound to ISG15.

63. The method of claim 48, wherein the first antibody is an immobilized antibody.

64. An antibody that selectively binds to an ISG15-conjugate, wherein the ISG15-conjugate comprises ISG15 and an ISG15 conjugated protein, wherein the antibody does not selectively bind to ISG15 alone, and wherein the antibody does not selectively bind to the ISG15 conjugated protein alone.

65. The antibody of claim 64, wherein the antibody selectively binds to an ISG15-conjugate selected from the group consisting of ISG15-phospholipase PLCγ1 conjugate, an ISG15-Jak1 conjugate, an ISG15-ERK1 conjugate, an ISG15-ERK2 conjugate, and an ISG15-Stat1 conjugate.

66. The antibody of claim 65, wherein the antibody is a monoclonal antibody.

67. A method to inhibit cell proliferation comprising contacting a cell with a composition comprising an ISG15-conjugate.

68. The method of claim 67, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

69. The method of claim 67, wherein the disorder related to cellular proliferation is cancer.

70. The method of claim 69, wherein the cancer is leukemia.

71. The method of claim 67, further comprising contacting the cell with retinoic acid.

72. A method to increase the intracellular concentration of an ISG15-conjugate comprising contacting the cell with a composition comprising the ISG15-conjugate.

73. The method of claim 72, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

74. A method to increase migration of a cell comprising contacting the cell, or a surface on which the cell migrates, with a composition comprising an ISG15-conjugate.

75. The method of claim 74, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

76. A method to increase phagocytotic activity of a cell comprising contacting the cell, or a surface on which the cell migrates, with a composition comprising an ISG15-conjugate.

77. The method of claim 76, wherein the cell is at the site of a wound.

78. The method of claim 76, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

79. A method to increase wound healing comprising contacting a wound with a composition comprising an ISG15-conjugate.

80. The method of claim 79, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

81. A method to determine the responsiveness of a patient to treatment with an interferon, comprising:
(a) administering an interferon to the patient suspected of being responsive to interferon treatment;
(b) determining the amount of ISG-15-conjugated protein in the patient; and
(c) comparing the amount of ISG-15-conjugated protein in the patient before and after administration of the interferon, wherein an increase in ISG15-conjugated protein indicates greater responsiveness to interferon treatment.

82. An ISG15-conjugate selected from the group consisting of ISG15-Stat1, ISG15-ERK1, ISG15-ERK2, ISG15-PLCγ1, and ISG15-Jak1.

83. An isolated complex comprising an ISG15-conjugate and an antibody that selectively binds to the conjugate.

84. The complex of claim 84, wherein the ISG15-conjugate is selected from the group consisting of an ISG15-phospholipase PLCγ1 conjugate, an ISG15-Jak1 conjugate, an ISG15-ERK1 conjugate, an ISG15-ERK2 conjugate, an ISG15-Stat1 conjugate, a variant thereof, and a combination thereof.

85. A method to increase the intracellular concentration of an ISG15-conjugate comprising contacting the cell with a composition comprising the compound identified according to the method of claim 25.

86. A method to inhibit cell proliferation comprising contacting a cell with a composition comprising the compound identified according to the method of claim 25.

87. A method to increase migration of a cell comprising contacting the cell, or a surface on which the cell migrates, with a composition comprising the compound identified according to the method of claim 25.

88. A method to increase phagocytotic activity of a cell comprising contacting the cell, or a surface on which the cell migrates, with a composition comprising the compound identified according to the method of claim 25.

89. A method to increase wound healing comprising contacting a wound with a composition comprising the compound identified according to the method of claim 25.


SEQUENCE LISTING (2 sequences)

SEQ ID NO: 1 - DNA, 26 bp, Artificial Sequence
  primer
tggaattcttaggcacactggtcccc

SEQ ID NO: 2 - DNA, 27 bp, Artificial Sequence
  primer
aattcgattctggatccgcctgggacc